(19) United States
(12) Patent Application Publication
(10) Pub. No.: US 2012/0065170 A1
(43) Pub. Date: Mar. 15, 2012

(54) Antimicrobial Cyclocarbonyl Heterocyclic Compounds For Treatment Of Bacterial Infections

(75) Inventors: Mikhail Fedorovich Gordeev, Castro Valley, CA (US); Jinqian Liu, Palo Alto, CA (US); Qiang Wang, Shanghai (CN); Li Zhang, Shanghai (CN)

(73) Assignee: MicuRx Pharmaceuticals, Inc., George Town (KY)

(21) Appl. No.: 13/228,332

(22) Filed: Sep. 8, 2011

Related U.S. Application Data
(60) Provisional application No. 61/381,804, filed on Sep. 10, 2010.

(51) Int. Cl. A61K 31/4439; A61P 31/04; A61K 31/675; C07D 413/14; C07F 9/09

(52) U.S. Cl. 514/89; 546/271.4; 514/340; 546/22

(57) ABSTRACT

The present invention provides indoline compounds of the following formula I:

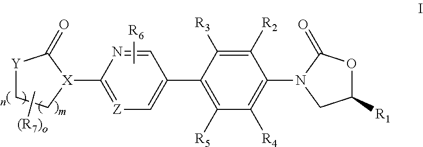

or pharmaceutically acceptable salts, prodrugs, solvates, or hydrates thereof useful as antibacterial agents, pharmaceutical compositions containing them, methods for their use, and methods for preparing these compounds.

### RELATED APPLICATIONS

This application claims the benefit of Provisional Patent Application Ser. No. 61/381,804, filed on 10 Sep. 2010, which application is incorporated in its entirety, as if fully set forth, herein.

### FIELD OF THE INVENTION

The present invention provides novel cyclocarbonyl (i.e. carbonyl-containing) heterocyclic compounds with useful antimicrobial properties, pharmaceutical compositions thereof, methods for their use, and methods for preparing of the same. These compounds have potent activity against various pathogenic bacterial species combined with a favorable tolerability profile.

### BACKGROUND OF THE INVENTION

Due to an increasing antibiotic resistance, novel classes of antibacterial compounds are acutely needed for the treatment of bacterial infections. The antibacterials should possess useful levels of activity against certain human and veterinary pathogens, including Gram-positive aerobic bacteria such as multiply-resistant Staphylococci and Streptococci, select anaerobes such as Bacteroides and Clostridia species, and acid-fast microorganisms such as Mycobacterium tuberculosis and Mycobacterium avium.

It is also important that such antibacterial agents should offer sufficient safety with a minimal toxicity and adverse effects that can preclude or limit the therapy.

Among newer antibacterial agents, oxazolidinone compounds are the most recent synthetic class of antimicrobials active against several key pathogenic microbes, including methicillin-resistant Staphylococcus aureus (MRSA). To date, a sole antibacterial of this class linezolid (Zyvox®) has been approved for a treatment of select Gram-positive infections.

While linezolid is widely used in antimicrobial therapy, its antibacterial activity is limited in two key aspects. First, its antibacterial spectrum (i.e. coverage) is generally limited to Gram-positive microorganisms, with no therapeutic activity against key Gram-negative infections. Thus, it has only modest activity against the fastidious Gram-negative pathogen Haemophilus influenzae, with typical MIC₉₀ (i.e. minimum inhibitory concentration for 90% of strain being tested) of 16 μg/mL. This value is well above useful MIC₉₀ for linezolid which is in the range of 2-4 μg/mL against Gram-positive Staphylococcus species for which the drug is indicated. Subsequently, linezolid is not prescribed for the treatment of infections caused by H. influenzae, which is an important causative pathogen in several serious infections, including certain types of pneumonia and bacterial meningitis. No oxazolidinone agent is presently approved for the treatment of H. influenzae infections.

Secondly, linezolid-resistant bacteria such as linezolid-resistant Enterococcus faecium and Staphylococcus aureus strains has been documented in recent years. Linezolid is not indicated for therapy of infections caused by linezolid-resistant bacterial strains, against which it displays MICs of 8 μg/mL and higher, since the drug is generally ineffective against such infections. Indeed, several deaths resulting from linezolid therapy failure in infections due to such resistant bacteria have been reported, for example, by Garcia et al. in J. Amer. Med. Association (JAMA), 2010, vol. 303, No. 22, p. 2260. Bacterial resistance is expected to become even more problematic with a continued linezolid use due to the continuous adaptation of microbial species, as reviewed, for example, by Walsh in Antibiotics: Actions, Origins, Resistance, 2003. For example, linezolid-resistance in multiple clones of S. aureus and Staphylococcus epidermidis has been recently reported by Wong et al. in Antimicrob. Agents Chemotherapy, 2010, vol. 54, No. 2, p. 742. Thus, newer agents with an improved potency and bacterial spectrum are urgently needed.

In over 10 years since the approval of the first drug of this class, linezolid (Zyvox®), numerous attempts to introduce second-generation oxazolidinone drugs with improved activity have been unsuccessful. It is recognized that this failure resulted mainly due to a frequently increased toxicity and poor tolerability of more potent but less selective oxazolidinones, as reviewed, for example, by Poce et al. in Expert Opin. Ther. Patent, 2008, vol. 12, No. 2, p. 97. Myelosuppression or bone marrow toxicity was reported as the chief factor limiting therapy of linezolid, as reflected in the warning included into Zyvox® prescribing information. Bone marrow suppression (also referred to as hematopoietic toxicity or myelosuppression) was reported, for example, by Monson et al. in Clinical Infectious Diseases, 2002, vol. 35, pp. e29-31. Additional adverse effects associated with ZyvoxR include anemia, leukopenia, pancytopenia, and thrombocytopenia. What is needed is next generation oxazolidinones that combine the aforementioned expanded antibacterial coverage and enhanced potency over linezolid together with therapeutically acceptable tolerability.

None of aforementioned publications specifically contemplates compounds of the present invention, their beneficial potency or safety profiles, their combination therapies, or their novel compositions.

### SUMMARY OF THE INVENTION

The present invention provides novel cyclocarbonyl (i.e. carbonyl-containing) heterocyclic oxazolidinone compounds with useful antibacterial activity. Within the scope of this invention, carbonyl-containing heterocyclic oxazolidinone compounds comprise a saturated non-aromatic carbonyl-containing heterocyclic ring connected to a phenyloxazolidinone fragment via a pyridine or pyrimidine aromatic linker, with said phenyloxazolidione containing either a substituted or an unsubstituted benzene fragment.

The activity for compounds of this invention includes antibacterial activity against Gram-positive microorganisms, such as Staphylococcus aureus, Staphylococcus epidermidis, Streptococcus pneumoniae, Enterococcus faecalis, and Enterococcus faecium, as well as against key linezolid-resistant pathogens, including linezolid-resistant Staphylococci, Streptococci, and Enterococci. These compounds are also active against fastidious Gram-negative pathogens, including H. influenzae and Moraxella catarrhalis. Furthemore, the compounds of present invention are also active against mycobacterial species, including Mycoplasma tuberculosis and Mycobacterium avium.

Surprisingly, certain compounds of the present invention are active against key multi-drug resistant bacteria, including MRSA, VRE, PRSP, and against linezolid-resistant Gram-positive bacteria, such as linezolid-resistant Enterococcus faecium, Enterococcus faecalis, and Staphylococcus aureus. Furthermore, certain compounds of the present invention are also active against fastidious Gram-negative pathogens, such as Haemophilus influenzae. The compounds provided herein are useful as antibacterial agents for treatment of infections including, but not limited to, skin infections, soft tissue infections, bacteremia, respiratory tract infections, urinary tract infections, bone infections, and eye infections.

As exemplified in the results described below, compounds provided herein combine the useful activity against multiple pathogens and expanded antibacterial spectrum with enhanced safety and tolerability, as compared to other antibacterial agents of the oxazolidinone class. Thus, the compounds of this invention offer a unique benefit of an enhanced therapy with a minimized potential for undesired adverse effects in human and animals.

The present invention provides a compound of the following formula I:

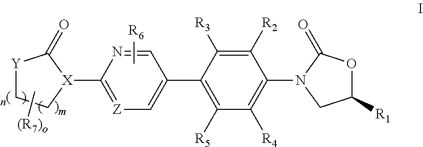

or a pharmaceutically acceptable salt, prodrug, solvate, or hydrate thereof wherein:

R¹ is CH₂OH, CH₂OPO₃H₂, CH₂F, CH₂NHC(═O)OC₁₋₅alkyl, (4-R⁸-1,2,3-triazol-1-yl)methyl, (5-R⁸-isoxazol-3-yl)aminomethyl, or (5-R⁸-isoxazol-3-yl)oxymethyl, wherein R⁸ is H, C₁₆alkyl, halo, or CN;

R² and R⁴ are independently H or F;

R³ and R⁵ are independently H, F, CN, or CH₃;

R⁶ is H, halo, or C₁₋₆alkyl;

R⁷ is a single or multiple substituent(s) selected from H, F, C₁₋₆alkyl, or C₃₋₆ cycloalkyl;

X is N, CH, or CF;

Y is NH, NC₁₋₄alkyl, O, CH₂, CHF, or CF₂;

Z is CH, CF, or N;

m, n, and o are independently 0, 1, or 2.

The alkyl, alkenyl, or cycloalkyl groups at each occurrence above independently are optionally substituted with one, two, or three substituents selected from the group consisting of halo, aryl, Het¹, and Het². Het¹ at each occurrence is independently a C-linked 5 or 6 membered heterocyclic ring having 1 to 4 heteroatoms selected from the group consisting of oxygen, nitrogen, and sulfur within the ring. Het² at each occurrence is independently a N-linked 5 or 6 membered heterocyclic ring having 1 to 4 nitrogen and optionally having one oxygen or sulfur within the ring.

In certain aspects of this invention, when R₁ is CH₂OH, CH₂OPO₃H₂, CH₂F, CH₂NHC(═O)OC₁₋₅alkyl, (4-R⁸-1,2,3-triazol-1-yl)methyl, (5-R⁸-isoxazol-3-yl)aminomethyl, or (5-R⁸-isoxazol-3-yl)oxymethyl, wherein R⁸ is H, C₁₋₆alkyl, halo, or CN; with a proviso that when X is N, and Y is O; then R⁷ is other than F or C₁₋₆alkyl.

In certain aspects of this invention, when R₁ is (4-R⁸-1,2,3-triazol-1-yl)methyl, (5—R⁸-isoxazol-3-yl)aminomethyl, or (5-R⁸-isoxazol-3-yl)oxymethyl, then at least one of R² and R⁴ is F.

In certain other aspects, when R₁ is (4-R⁸-1,2,3-triazol-1-yl)methyl, (5-R⁸-isoxazol-3-yl)aminomethyl, or (5-R⁸-isoxazol-3-yl)oxymethyl; X is N; and Y is O; then R⁷ is H.

In certain aspects, when R₁ is CH₂OH, CH₂OPO₃H₂, CH₂F, (4-R⁸-1,2,3-triazol-1-yl)methyl, (5-R⁸-isoxazol-3-yl)aminomethyl, or (5-R⁸-isoxazol-3-yl)oxymethyl; X is N; and Y is O; then R⁷ is H, or o is 0.

In certain aspects, R¹ in a compound of formula I is CH₂OH or CH₂OPO₃H₂, and R⁷ is H or F.

In certain other aspects, provided herein is an ester derivative of the compound of formula I, wherein R¹ is CH₂O(C═O)-alkylamine or CH₂O(C═O)-cycloalkylamine. Exemplary alkylamine groups include, for example, respective groups of amino acids alanine, valine, isoleucine, leucine, glycine, or alike. Exemplary cycloalkylamine groups include, for example, respective groups of amino acids proline, pipecolic acid, or alike.

In certain aspects, R¹ in a compound of formula I is R¹ is (4-R⁸-1,2,3-triazol-1-yl)methyl, (5-R⁷-isoxazol-3-yl)aminomethyl or (5-R⁸-isoxazol-3-yl)oxymethyl, wherein R⁸ is H, C₁₋₃alkyl, halo, or CN.

In certain aspects, R² and R⁴ are H; and R³ and R⁵ are independently selected from H and F.

In certain other aspects, R¹ is CH₂OH or CH₂OPO₃H₂, X is N; Y is CH₂, CHF, CF₂, or O; and R⁷ is H.

In certain aspects, R² and R⁴ are H; and R³ and R⁵ are independently selected from H and F.

In certain aspects, R¹ is CH₂OH or CH₂OPO₃H₂; m and n are both 1; and o is 0.

In another aspect, the present invention provides a pharmaceutical composition comprising a compound of any of formula I, or a pharmaceutically acceptable salt thereof, and a pharmaceutically acceptable carrier.

In another aspect, the present invention provides a method for treating microbial infection in a mammal by administering to the mammal in need a therapeutically effective amount of a compound of any of formula I or a pharmaceutically acceptable salt thereof.

In certain aspects, the microbial infection is a Gram-positive microbial infection.

In certain aspects, the microbial infection is a Gram-positive microbial infection caused by linezolid-resistant bacteria.

In certain aspects, the microbial infection is a fastidious Gram-negative microbial infection.

In certain aspects, the microbial infection is a mycobacterial infection, including tuberculosis.

The compounds of formula I may be administered orally, parenterally, transdermally, topically, rectally, or intranasally.

The compounds of formula I may be administered once-daily in an amount of from about 1 to about 75 mg/kg of body weight/day.

In certain aspects, provided herein is a compound according to any one of formula I for use in therapy.

In certain aspects, provided herein is a compound according to any one of formula I for use in the treatment of a microbial infection in a mammal in need thereof.

In certain aspects, provided herein is use of a compound according to any one of formula I in the manufacture of a medicament for therapy.

In certain aspects, provided herein is use of a compound according to any one of formula I in the manufacture of a medicament for treatment of a bacterial infection in a mammal in need thereof. In another aspect, the compounds of formula I can be used in combinations with other bioactive agents, such as anti-infective or anti-inflammatory agents. For example, to achieve an optimal therapeutic effect (such as a broad spectrum of action), compounds of formulas I may be co-administered in a combination with an antimicrobial agent active against non-fastidious Gram-negative bacteria (e.g., quinolone, beta-lactam, aminoglycoside, colistin, macrolide agent, etc.), an agent active against pathogenic fungi or yeast (e.g., allylamine, terbinafine, azole, etc.), or in combination with an antiviral agent (such as an entry-blocker, viral protease or DNA inhibitor, antiretroviral agent, etc.).

In yet another aspect, the present invention provides certain novel intermediates and processes for preparing compounds of formula I.

### DETAILED DESCRIPTION OF THE INVENTION

Unless otherwise stated, the following terms used in the specification and Claims have the meanings given below.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive. Group R^{#} is same as R_{#}: R¹ is same as R₁, etc.

The terms “alkyl,” “alkenyl,” etc. refer to both straight and branched groups, but reference to an individual radical such as “propyl” embraces only the straight chain radical, a branched chain isomer such as “isopropyl” being specifically referred to. The alkyl, alkenyl, etc., group may be optionally substituted with one, two, or three substituents selected from the group consisting of halo, aryl, Het¹ or Het². Representative examples include, but are not limited to, difluoromethyl, 2-fluoroethyl, trifluoroethyl, —CH═CH-aryl, —CH═CH-Het¹, —CH₂-phenyl, and the like.

The term “cycloalkyl” means a cyclic saturated monovalent hydrocarbon group of three to six carbon atoms, e.g., cyclopropyl, cyclohexyl, and the like. The cycloalkyl group may be optionally substituted with one, two, or three substituents selected from the group consisting of halo, aryl, Het¹ or Het².

The term “heteroalkyl” means an alkyl or cycloalkyl group, as defined above, having a substituent containing a heteroatom selected from N, O, or S(O)ₙ, where n is an integer from 0 to 2, including, hydroxy (OH), C₁₋₄alkoxy, amino, thio (—SH), and the like. Representative substituents include —NRₐR_{b}, —ORₐ, or —S(O)ₙR_{c}, wherein Rₐ is hydrogen, C₁₋₄alkyl, C₃₋₆cycloalkyl, optionally substituted aryl, optionally substituted heterocyclic, or —COR (where R is C₁₋₄alkyl); R_{b} is hydrogen, C₁₋₄alkyl, —SO₂R (where R is C₁₋₄alkyl or C₁₋₄hydroxyalkyl), —SO₂NRR′ (where R and R′ are independently of each other hydrogen or C₁₋₄alkyl), —CONR′R″ (where R′ and R″ are independently of each other hydrogen or C₁₋₄alkyl); n is an integer from 0 to 2; and R_{c} is hydrogen, C₁₋₄alkyl, C₃₋₆cycloalkyl, optionally substituted aryl, or NRₐR_{b} where Rₐ and R_{b} are as defined above. Representative examples include, but are not limited to, 2-methoxyethyl (—CH₂CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to phenyl, biphenyl, or naphthyl, optionally substituted with 1 to 3 substituents independently selected from halo, —C₁₋₄alkyl, —OH, —OC₁₋₄alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or —C₁₋₄alkyl. Likewise, the term phenyl refers to the phenyl group optionally substituted as above.

The term “heterocyclic ring” refers to an aromatic ring or a saturated or unsaturated ring that is not aromatic of 3 to 10 carbon atoms and 1 to 4 heteroatoms selected from the group consisting of oxygen, nitrogen, and S(O)ₙ within the ring, where n is defined above. The heterocyclic ring may be optionally substituted with halo, —C₁₋₄alkyl, —OH, —OC₁₋₄ alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or C₁₋₄alkyl.

Examples of heterocylic rings include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiadiazole tetrazole, thiazolidine, thiophene, benzo[b]thiophene, morpholinyl, thiomorpholinyl (also referred to as thiamorpholinyl), piperidinyl, pyrrolidine, tetrahydrofuranyl, 1,3-benzoxazine, 1,4-oxazine-3-one, 1,3-benzoxazine-4-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one and the like. Heterocyclic rings include unsubstituted and substituted rings.

Specifically, Het¹ (same as het¹, Het₁ or het₁) refers to a C-linked five-(5) or six-(6) membered heterocyclic ring, including bicyclic rings. Representative examples of “Het¹” include, but are not limited to, pyridine, thiophene, furan, pyrazole, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, 1,2,4-dithiazolone, or 3-azabicyclo[3.1.0]hexan-6-yl.

Het² (same as het², Het₂, or het₂) refers to an N-linked five-(5) or six-(6) membered heterocyclic ring having 1 to 4 nitrogen atoms, and optionally having one oxygen or sulfur atom, including bicyclic rings. Representative examples of “Het²” include, but are not limited to pyrrolyl, imidazolyl, pyrazolyl, 1,2,3-triazolyl, 1,2,4-triazolyl, 1,2,3,4-tetrazolyl, isoxazolidinonyl group, 3-azabicyclo[3.1.0]hexan-3-yl, 1,3,9,9a-tetrahydrooxazolo[3,4-a]indol-1-yl, 2-alkylpyrrolo[3,4-c]pyrazol-5(2H,4H,6H)-yl, and 5H-pyrrolo[3,4-b]pyridin-6(7H)-yl.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center as determined using the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and Claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A hydrogen (H) or carbon (C) substitution for compounds of the formula I include a substitution with any isotope of the respective atom. Thus, a hydrogen (H) substitution includes a ¹H, ²H (deuterium), or ³H (tritium) isotope substitution, as may be desired, for example, for a specific therapeutic, diagnostic therapy, or metabolic study application. Optionally, a compound of this invention may incorporate a known in the art radioactive isotope or radioisotope, such as ³H, ¹⁵O, ¹⁴C, or ¹³N isotope, to afford a respective radiolabeled compound of formula I.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.
A “therapeutically effective amount” means the amount of a compound that, when administered to a mammal for treating a disease, is sufficient to effect such treatment for the disease. The therapeutically effective amount will vary depending on the compound, the disease and its severity and the age, weight, etc., of the mammal to be treated. Therapeutically effective amount may also be referred to as any amount of a compound that is sufficient to achieve the desired beneficial effect, including preventing the disease, inhibiting the disease, or relieving the disease, as described above in (1)-(3). For example, the amount of a compound can range between 0.1-250 mg/kg, or preferably, 0.5-100 mg/kg, or more preferably, 1-50 mg/kg, or even more preferably, 2-20 mg/kg. More preferably, said amount of a compound is administered to a mammal once-daily. Even more preferably, said amount of a compound is administered to a mammal once-weekly or once-biweekly.

“Leaving group” has the meaning conventionally associated with it in synthetic organic chemistry, i.e., an atom or group capable of being displaced by a nucleophile and includes halogen, C₁₋₄alkylsulfonyloxy, including but not limited to chloro, bromo, iodo, mesyloxy, tosyloxy, trifluorosulfonyloxy, and the like.

“Prodrug” means any compound which releases an active parent drug according to a compound of the subject invention in vivo when such prodrug is administered to a mammalian subject. Various prodrugs have been described, for example, in the following publications: Alexander et al. J. Med. Chem. 1988, p. 318; Alexander et al. J. Med. Chem., 1991, p. 78; Murdock et al. J. Med. Chem., 1993, p. 2098; Davidsen et al. J. Med. Chem., 1994, p. 4423; Robinson et al. J. Med. Chem., 1996, p. 10; Keyes et al. J. Med. Chem., 1996, p. 508; Krise et al. J. Med. Chem., 1999, p. 3094; Rahmathullah et al. J. Med. Chem., 1999, p. 3994; Zhu et al. Bioorg. Med. Chem. Lett., 2000, p. 1121; Sun et al., J. Med. Chem., 2001, p. 2671; Ochwada et al., Bioorg. Med. Chem. Lett., 2003, p. 191; Sohma et al. Med. Chem., 2003, p. 4124; Ettmayer et al. J. Med. Chem., 2004, p. 2393; Stella et al., Adv. Drug Delivery Rev., 2007, p. 677, Josyula et al. International Patent Publication No. WO 2005/028473; Rhee et al. International Patent Publication No. WO 2005/058886, and EP 1,683,803. Following the methods of these publications and references cited therein, prodrugs of the compounds of the present invention can be likewise prepared. Thus, prodrugs of compounds of the formula I are prepared by modifying functional groups present in a compound of the subject invention in such a way that the modifications may be cleaved in vivo to release the parent compound. Said prodrugs can be used, for example, to improve aqueous solubility, oral, transdermal, or ocular bioavailability, to achieve a controlled (e.g., extended) release of the drug moiety, to improve tolerability, etc. Prodrugs include compounds of the subject invention wherein a hydroxy, sulfhydryl, amido or amino group in the compound is bonded to any group that may be cleaved in vivo to regenerate the free hydroxyl, amido, amino, or sulfhydryl group, respectively. Examples of prodrugs include, but are not limited to esters (e.g., acetate, formate, benzoate, phosphate or phosphonate derivatives), carbamates (e.g., N,N-dimethylaminocarbonyl), N-phosphoramides, of hydroxyl or amine-derived functional groups in compounds of the subject invention. Prodrug derivative can be used either as a neutral prodrug form (e.g. acid or amine), or a respective salt form thereof [e.g. sodium salt of a phosphate prodrug, or an amine salt (e.g. hydrochloride, citrate, etc.) for an amine group-bearing prodrug], or a zwitterionic form if both positively and negatively charged/ionizable functions are present. Prodrug groups may be incorporated at various sites of the formula I, provided that at least one appropriate functionality is available for a prodrug group installation.

Several preferred prodrug structures of this invention are illustrated below.

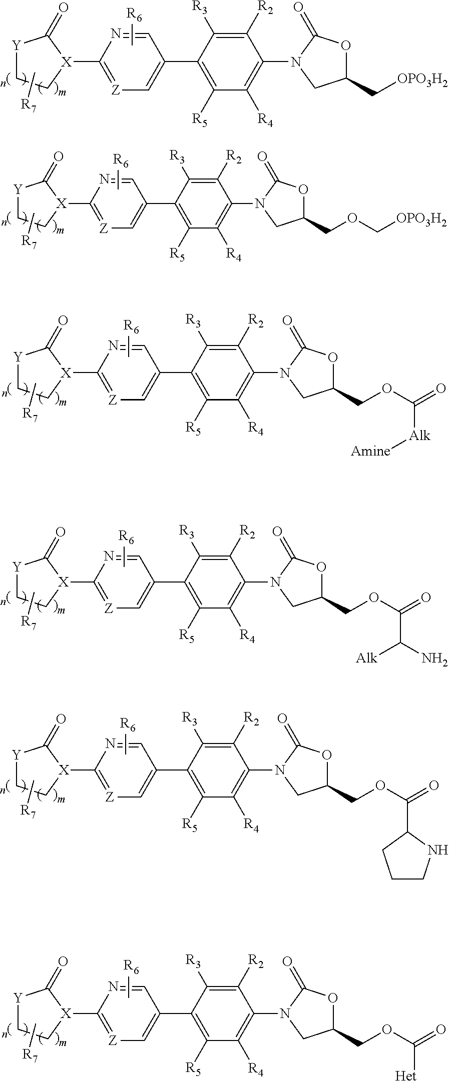

Additional preferred prodrug structures of this invention are illustrated below.

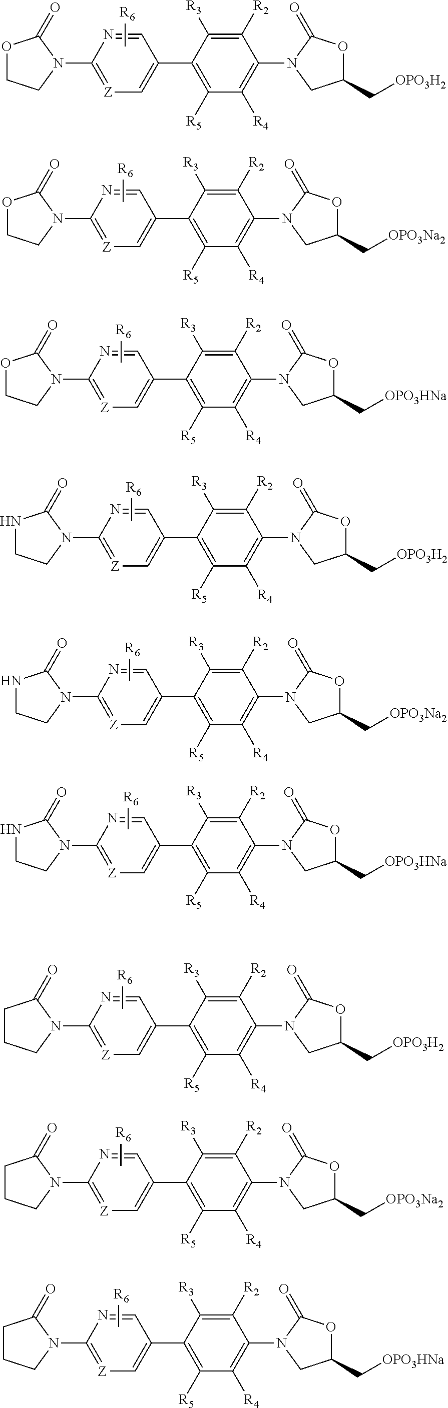

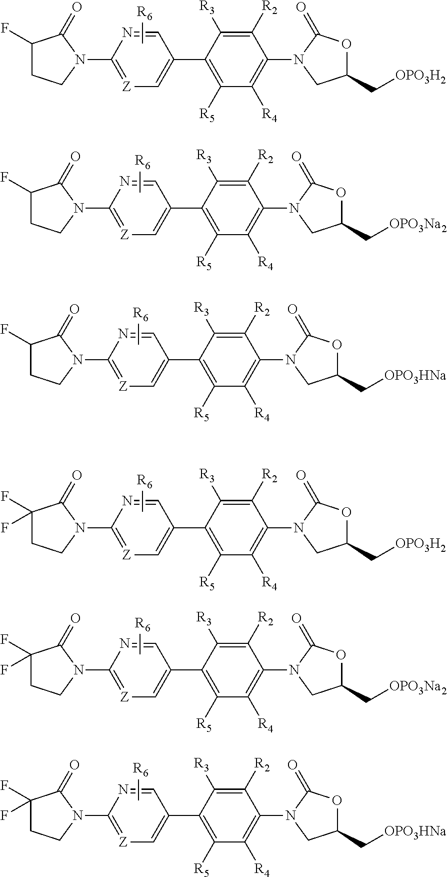

The term “mammal” refers to all mammals including humans, livestock, and companion animals.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “r.t.” for room temperature).

### Illustrative Aspects

Within the broadest definition of the present invention, certain compounds of the compounds of formula I may be preferred. Specific and preferred values listed below for radicals, substituents, and ranges, are for illustration only; they do not exclude other defined values or other values within defined ranges for the radicals and substituents.

In some preferred compounds of the present invention C₁₋₄alkyl can be methyl, ethyl, propyl, isopropyl, butyl, iso-butyl, sec-butyl, and isomeric forms thereof.

In some preferred compounds of the present invention C₂₋₄alkenyl can be vinyl, propenyl, allyl, butenyl, and isomeric forms thereof (including cis and trans isomers).

In some preferred compounds of the present invention C₃₋₆cycloalkyl can be cyclopropyl, cyclobutyl, cyclopentyl, cyclohexyl, and isomeric forms thereof.

In some preferred compounds of the present invention C₁₋₄ heteroalkyl can be hydroxymethyl, hydroxyethyl, and 2-methoxyethyl.

In some preferred compounds of the present invention halo can be fluoro (F) or chloro (Cl).

In some preferred compounds of the present invention R¹ can be CH₂NHC(═O)OC₁₋₅alkyl.

In some preferred compounds of the present invention R¹ can be (4-R⁸-1,2,3-triazol-1-yl)methyl, (5-R⁷-isoxazol-3-yl)aminomethyl, or (5-R⁸-isoxazol-3-yl)oxymethyl, wherein R⁸ is H, C₁₋₃alkyl, halo, or CN.

In some preferred aspects, group R¹ is selected from CH₂OH and CH₂OPO₃H₂.

In some preferred aspects, group R¹ is CH₂NHC(═O)OMe.

In some preferred aspects, group R¹ is selected from CH₂(1,2,3-triazol-1-yl) or CH₂(4-methyl-1,2,3-triazol-1-yl).

In some preferred aspects, group R¹ is selected from CH₂NH(isoxazol-3-yl) or CH₂O(isoxazol-3-yl).

In some preferred aspects, groups R², R³, R⁴ and R⁵ are independently selected from H or F.

In some preferred aspects, group R² is H, and group R⁴ is F.

In some preferred aspects, R², R³ and R⁴ are H, and R⁵ is F.

It will also be appreciated by those skilled in the art that compounds of the present invention may have additional chiral centers and be isolated in optically active and racemic forms. The present invention encompasses any racemic, optically active, tautomeric, or stereoisomeric form, or mixture thereof, of a compound of the invention.

One preferred group of compounds of the present invention is illustrated by the following structures.

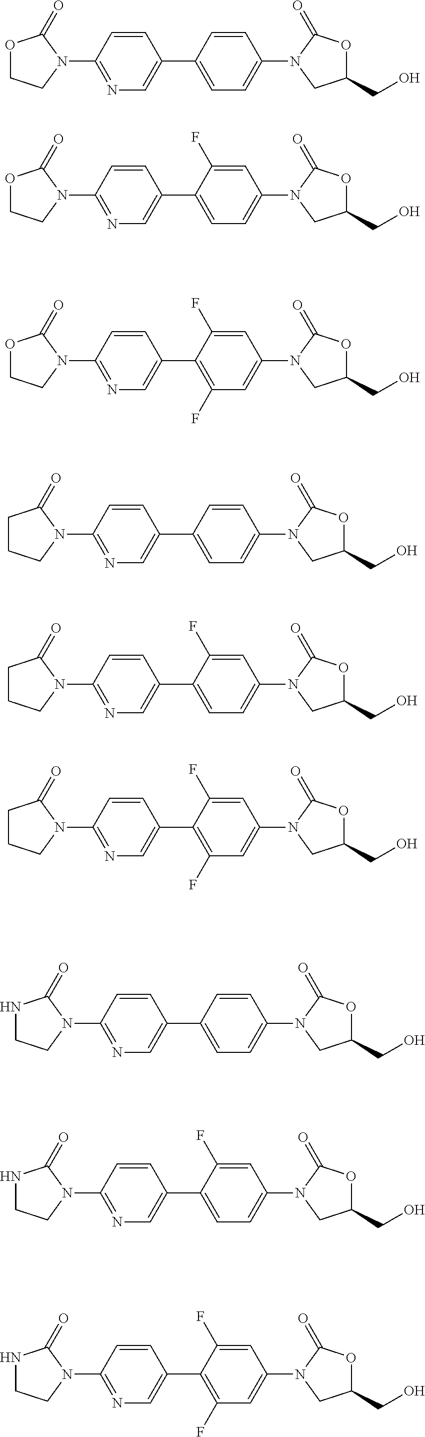

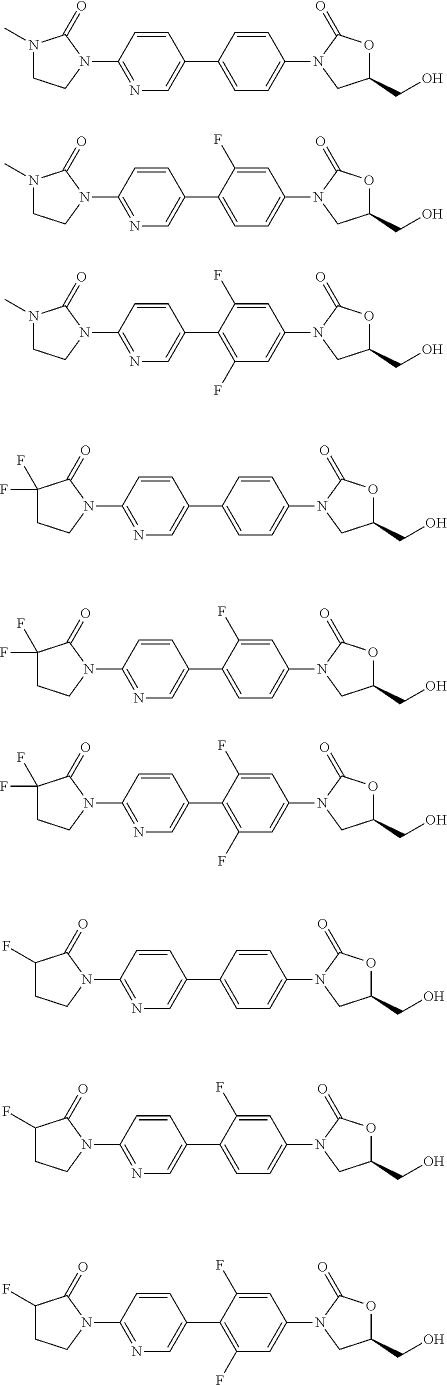

Additional preferred group of compounds of the present invention is illustrated by illustrated by the following structures.

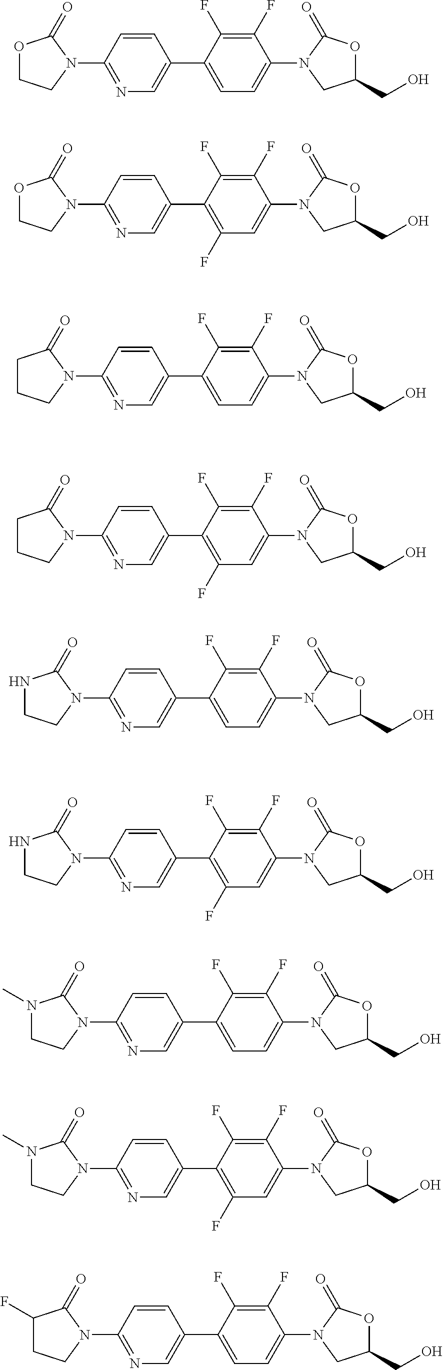

Another preferred group of compounds of the present invention is illustrated by the following structures.

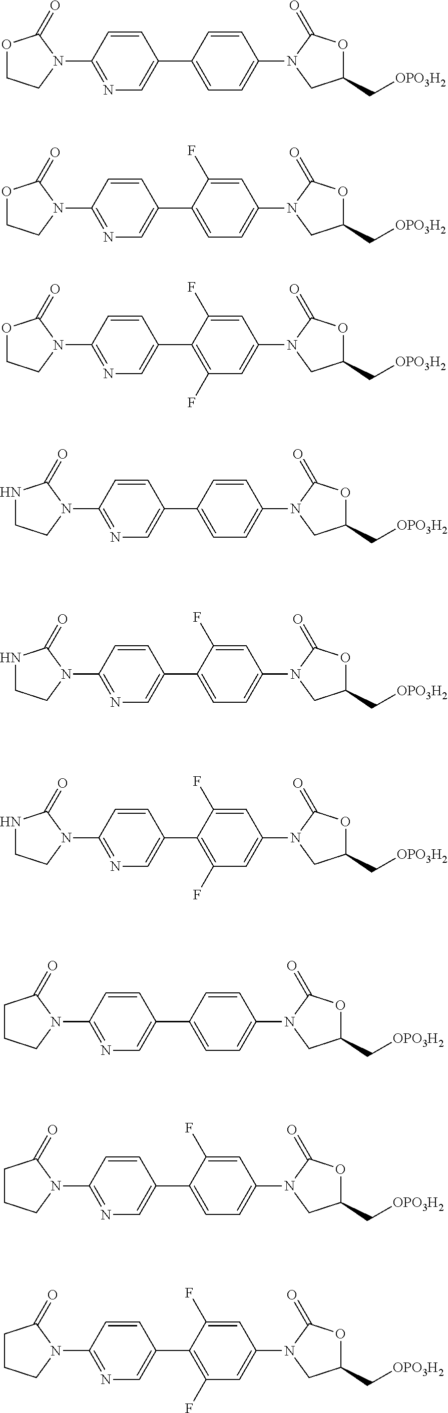

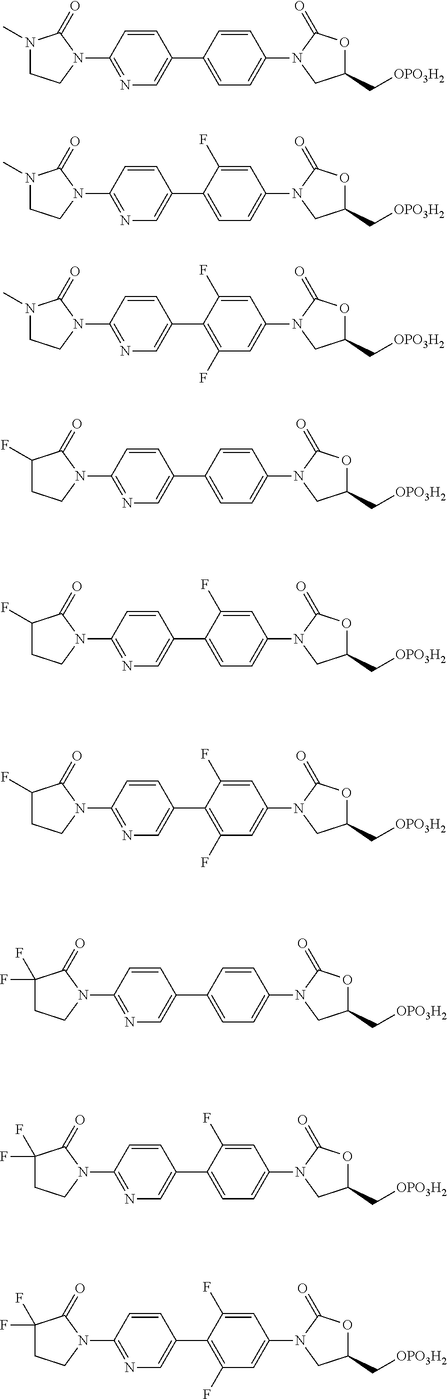

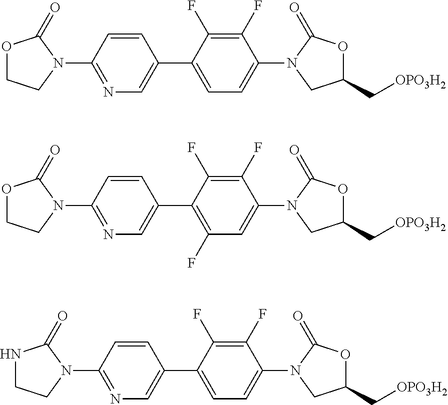

Another preferred group of compounds of the present invention is illustrated by the following structures.

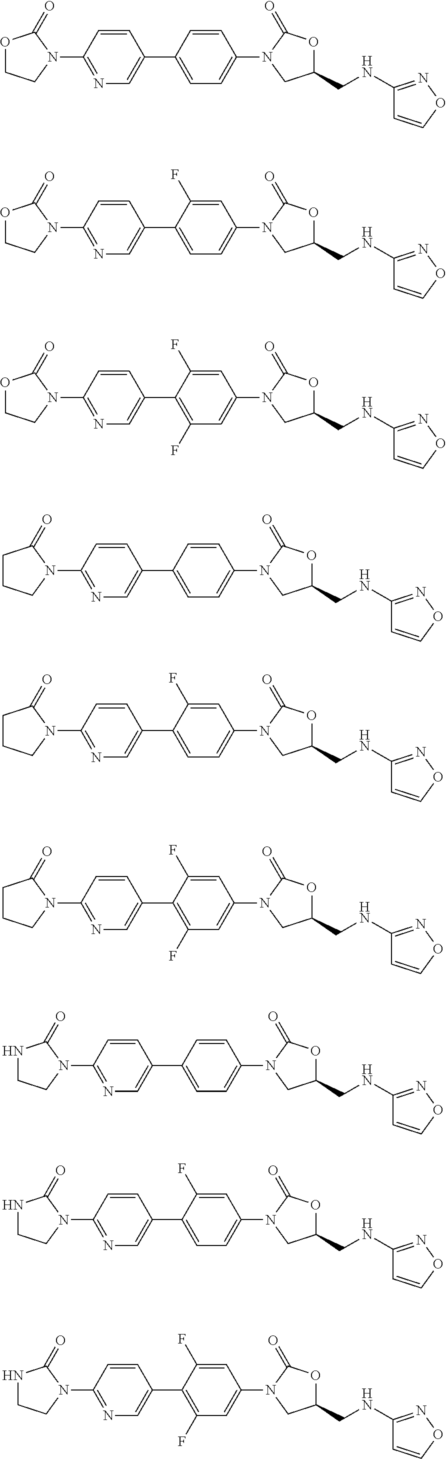

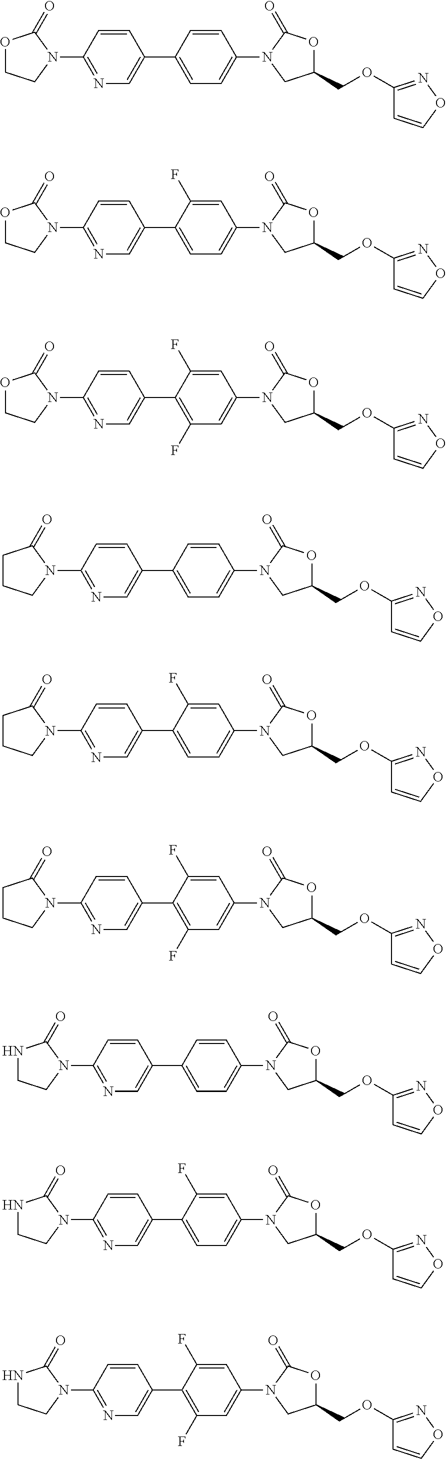

Another preferred group of compounds of the present invention is illustrated by the following structures.

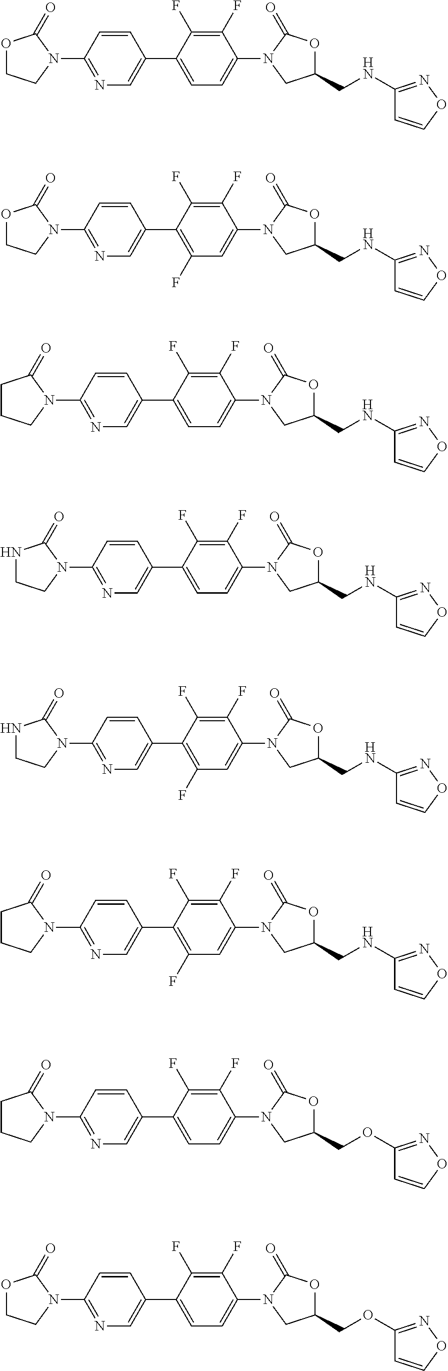

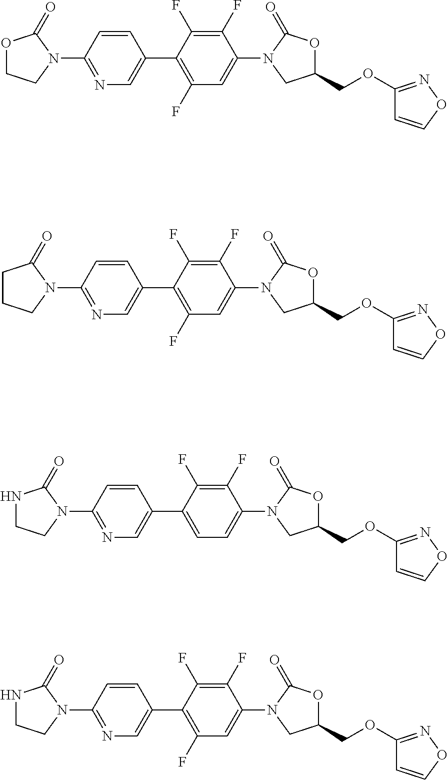

Another preferred group of compounds of the present invention is illustrated by the following structures.

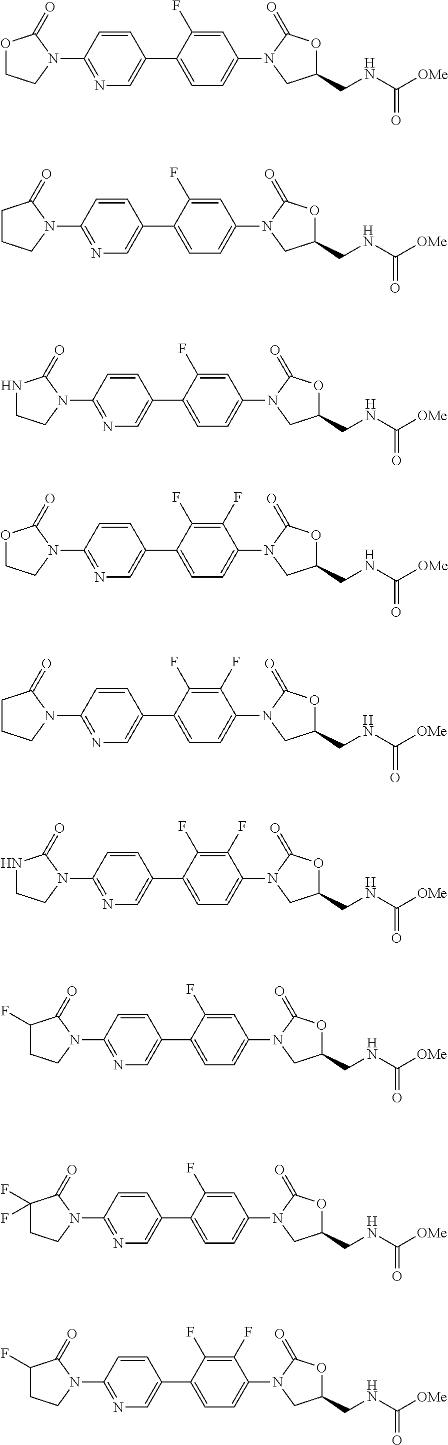

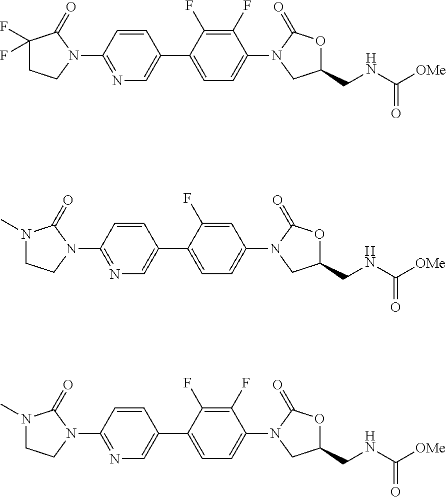

### General Synthetic Methods

The compounds of this invention can be prepared in accordance with one or more of Schemes discussed below.

One general approach to the compounds of this invention is illustrated in general Scheme 1. Synthetic steps of illustrative Schemes below have a relevant analogy in the general organic chemistry art.

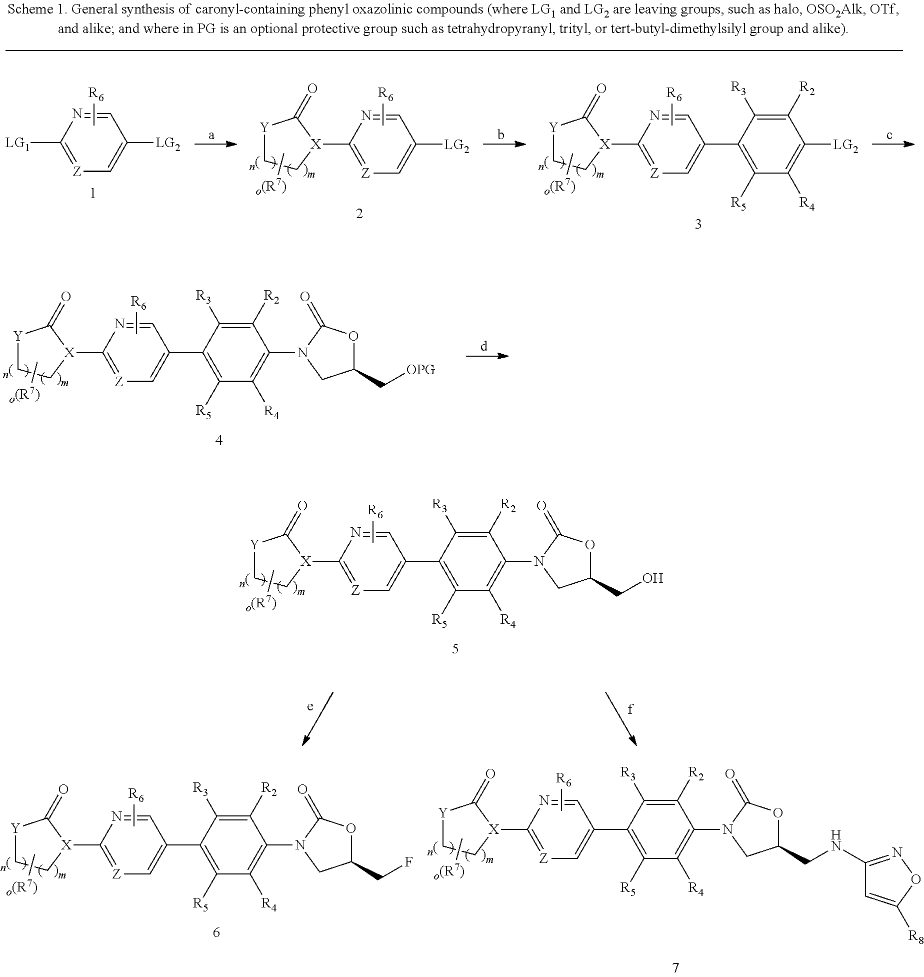

a) Carbonyl ring forming agent(s): base (Py, triethylamine (TEA), Na₂CO₃ or alike); base, and a catalytic metal (Pd), metallic compound (CuO, Ag₂O); b) arylating or heteroarylating reagent(s): Ar—B(OH)₂, or Ar—B(OAlk')₂ or Het¹-B(OH)₂, or Het¹-B(OAlk′)₂, or Het²-B(OH)₂, or Het²-B(OAlk′)₂ selected from boronic acid, boronic acid ester (e.g. (picolinato)boron ester) or alike, Pd catalyst [e.g. PdCl₂(dppf)DCM, Pd(PPh₃)₄ or alike]; c) oxazolinic ring forming agent(s): base (LiHMDS, or alike); Pd catalyst [e.g. PdCl₂(dppf)DCM, Pd(PPh₃)₄ or alike]; d) hydrogenation, or hydrolytic agent(s): H₂/Pd/C, ammounium formate/Pd, or alike; base (NaOH aqueous, alcoholic solution), or alike; e) Fluorinating agents: DAST; f) i) AlkylSO₂Cl/DCM, or THF, base (TEA, Na₂CO₃, or alike); ii) 3-(N-Boc-amino)-5-R⁸-isoxazole; base: e.g., NaH, LiOBu-t, KOBu-t, tetramethylguanidine, or alike; iii) acid: TFA or HCl solution in organic solvent, e.g., THF or dioxane); then base: NaHCO₃, TEA, or alike.

Methods for step (a) of Scheme 1 may involve one direct transformation, or several reactions to form the carbonyl-containing structure as illustrated in Scheme 2. When X is N, and LG₁ is halo, step a) may require an optional metal catalysis (such as Pd(dppf)Cl₂ DCM) or a metal compound (such as Pd, CuO), when LG₁ is a N-containing group, the transformation may be accomplished with a base under ambient or elevated temperature, as needed.

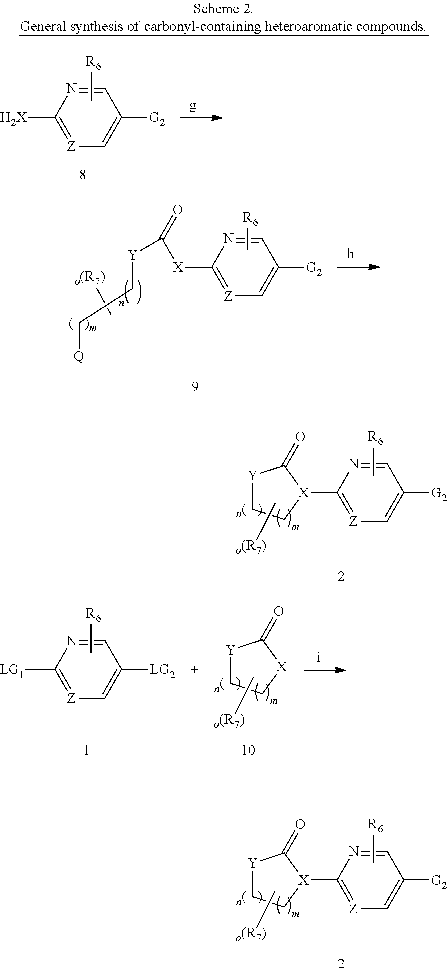

g) Acylating or coupling agent(s): base (Py, triethylamine (TEA), Na₂CO₃ or alike); HOBT, EDC, HBTU, or alike in a solvent (DCM, THF, DMF); h) Cyclizing agent(s), base (ammonia, NaH, LiHMDS, TEA, Na₂CO₃, or alike); c) arylating or heteroarylating reagent(s): Pd catalyst [e.g. PdCl₂(dppf)DCM, Pd(PPh₃)₄ or alike].

Methods for metal-mediated arylation of step (b) of Scheme 1 have been more generally reviewed, for example, in Synthesis, 2004, p. 2419. The boron coupling chemistry illustrated for above step (b) may be supplanted by other metal-mediated couplings, such as tin-coupling chemistry similar to that more generally described, for example, in Tetrahedron Lett., 1988, p. 2135.

Another general synthesis of compounds of the present invention is illustrated by Scheme 3. The oxazolidine structure may be formed in the beginning of the sequence, and the carbonyl-containing ring formed at the end of the transformations.

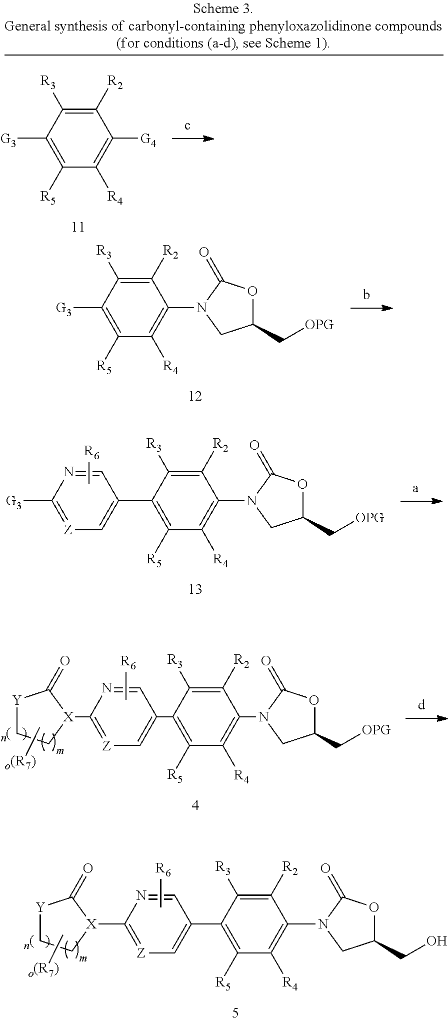

Intermediate 4 can be formed directly from reagents 2 and 7 as illustrated by Scheme 4.

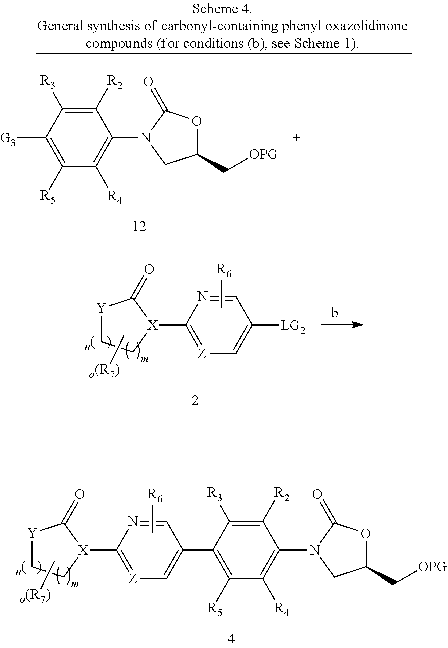

This invention also provides the methods for the synthesis of phosphate derivatives of the carbonyl-containing phenyloxazolidinone compounds as illustrated in Scheme 5.

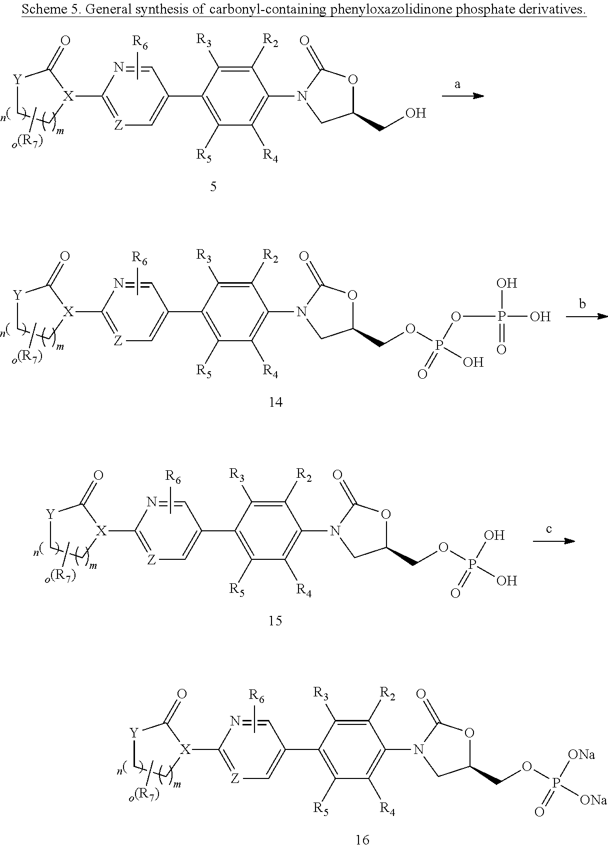

a) pyrophosphoryl monochloride, temperature from −20° C. to 50° C., in a solvent e.g. THF, DCM, MeCN; b) water; c) 2.0 eq. base, e.g., Na₂CO₃, NaHCO₃, or NaOH, or alike.

The phosphate derivatives of this invention can be also generally prepared as illustrated in general Scheme 6.

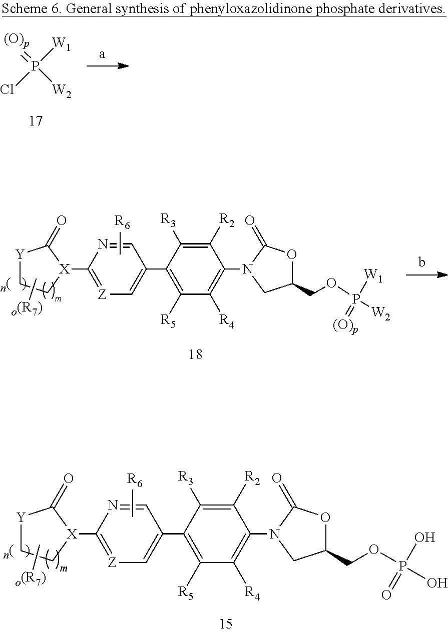

a) Compound 5; chlorophosphoryl reagent 17 (wherein W₁ and W₂ are independent leaving groups, including halo, phosphate, or OH) such as POCl₃, or PCl₃, temperature from −50° C. to 50° C., base, e.g., NaOH, trialkylamine, pyridine, imidazole, trialkylphosphate (e.g., (trimethyl or triethylphosphate), in a solvent e.g. THF, DCM, ACN; b) water.

The phosphate can be prepared either as mono- or bis-metal phosphate (Scheme 7), as needed. For example, if 1.0 eq. of metal base is used the mono-phosphate (such as monosodium phosphate) is obtained, whereas 2.0 eq. base results in bis-metal phosphate such as disodium phosphate. As needed, mono-alkyl mono-phosphate can be obtained likewise from respective mono-alkyl phosphate ester derivative of the compound 15.

The phosphorus-containing reagent in Schemes 5 and 6 can be modified before use or directly in the reaction medium (i.e., in situ) without departing from the spirit and scope of this invention. For example, POCl₃ can be modified with a base (such as trialkylamine, imidazole, pyridine, trialkyl phosphate) to a phosphoryl chloride intermediate. Pyrophosphoryl tetrachloride can be hydrolyzed in situ to pyrophosphoryl trichloride, which in turn to pyrophosphoryl dichloride, to pyrophosphoryl monochloride, as desired for a specific experimental procedure.

Additional detailed synthetic schemes for the syntheses of specific compounds of the present invention are illustrated by methods described for Examples below.

### EXAMPLES

Embodiments of the present invention are described in the following examples, which are meant to illustrate and not limit the scope of this invention. Common abbreviations well known to those with ordinary skills in the synthetic art are used throughout. Where applicable, compounds are named using IUPAC convention and SymyxDraw software. ¹H NMR spectra (δ, ppm) are recorded using 400 MHz NMR spectrometer in DMSO-d₆ unless specified otherwise. Mass-spectroscopy data for a positive ionization method are provided as obtained on LCMS spectrometer using trifluoroacetic acid (TFA) containing aqueous MeCN eluents. Chromatography means silica gel chromatography unless specified otherwise. TLC means thin-layer chromatography. Unless specified otherwise, all reagents were either from commercial sources, or made by conventional methods described in available literature.

### Example 1

### Compound of Structure

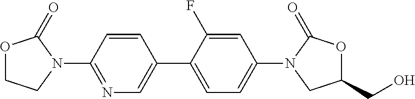

Scheme for Compound of Example 1:

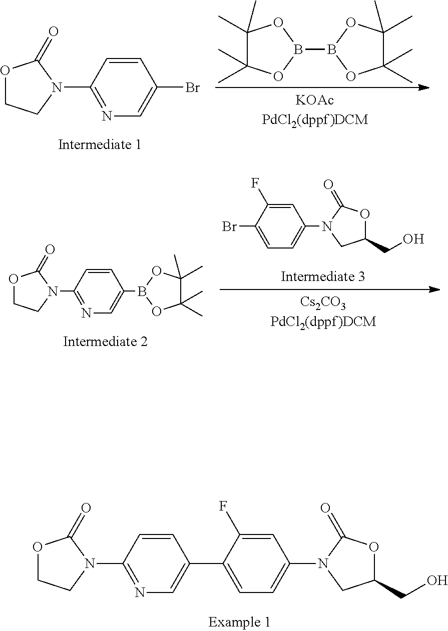

Intermediate 2. DMSO (10 mL) was added to the mixture of Intermediate 1 (2.0 g, 8 mmol, prepared as described in the publication PCT WO 2008/108988), bis(pinacolato)diboron (3.1 g, 12 mmol), KOAc (2.4 g, 24 mmol) and PdCl₂(dppf)DCM (300 mg, 0.4 mmol), and the reaction mixture was degassed with nitrogen for 30 min. The reaction mixture was heated at ca. 75° C. and stirred o.n. Water (500 mL) was added, and the resulting solid was filtered and washed with hexanes and DCM. The title compound was obtained as a brown solid. MS (m/z): 291 [M+H].

Compound of Example 1. DMF (10 mL) was added to the mixture of Intermediate 2 (1.0 g, 3.4 mmol), Intermediate 3 (1.0 g, 3.4 mmol, prepared analogously to the publication US 2003/0013737), Cs₂CO₃ (1.1 g, 3.4 mmol) and PdCl₂(dppf)DCM (128 mg, 0.17 mmol). The reaction mixture was degassed for 30 min, and then heated at ca. 55° C. for 2.5 h. EtOAc (400 mL) was added, insolubles were filtered off and washed with an excess of EtOAc. The organic layer was washed with brine and dried (Na₂SO₄). Solvent was removed and the residue was purified by silica gel column chromatography (eluent: gradient 3-5% MeOH in DCM). The title compound was obtained as a white solid. ¹H NMR: 8.57 (s, 1H); 8.18 (d, J=9.2 Hz, 1H); 8.04 (t, J=8.0 Hz, 1H); 7.67 (m, 2H); 7.48 (dd, J=8.4, 2.4 Hz 1H); 5.26 (s, 1H); 4.75 (m, 1H); 4.49 (t, J=8.0 Hz, 2H); 4.22 (t, J=8.0 Hz, 2H); 4.14 (t, J=9.2 Hz, 1H); 3.88 (dd, J=8.8, 6.4 Hz, 1H); 3.69 (d, J=3.2 Hz, 1H); 3.59 (d, J=3.2 Hz, 1H). MS (m/z): 374 [M+H].

### Example 2

### Compound of Structure

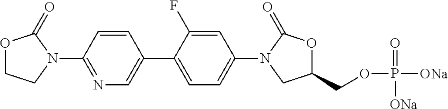

Scheme for Compound of Example 2:

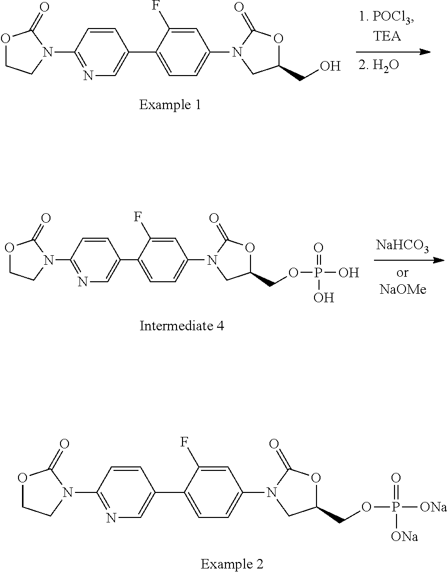

Intermediate 4. The compound of Example 1 (100 mg, 0.27 mmol) was dissolved in 15 mL of THF and cooled to 0° C. Et₃N (0.12 mL, 0.8 mmol) was added, followed by POCl₃ (0.074 mL, 0.8 mmol). The reaction mixture was stirred for 3.5 h at 0° C. Water (5 mL) was added and the mixture was stirred for another hour. After evaporation of the solution at 45° C., water (30 mL) was added and the resulted precipitate was filtered off and washed with H₂O and Et₂O. The solid was re-dissolved in EtOH (8 mL) and treated with activated carbon (50 mg). The mixture was filtered and the filtrate was evaporated to afford the title compound as a white solid. NMR: 8.57 (s, 1H); 8.19 (d, J=4.4 Hz, 1H); 8.04-8.07 (m, 1H); 7.54-7.68 (m, 2H); 7.46-7.49 (dd, J=8.8, 5.0 Hz, 1H); 4.94-4.95 (m, 1H); 4.50 (t, J=8.0 Hz, 2H); 4.20-4.27 (m, 3H); 4.05-4.12 (m, 2H); 3.89-3.92 (dd, J=9.2, 6.0 Hz, 1H). MS (m/z): 454.

Compound of Example 2.

Method A. 0.4 M Aq. NaHCO₃ (2.3 mL, 0.93 mmol) was added with stirring to Intermediate 4 (210 mg, 0.46 mmol) in EtOH (3 mL). The reaction mixture was stirred at r.t. for 2.5 h. Solvent was evaporated under vacuum at 45° C. and water (200 mL) was added. The mixture was filtered, and the precipitate washed with an excess of EtOH. Most volatiles were removed under vacuum and then EtOH—H₂O (5:1, 500 mL) was added. The mixture was stirred for 10 min and filtered off. The solid was washed with EtOH, DCM and Et₂O and dried under vacuum to afford the title compound as a white solid. MS (m/z): acid form ion 454 [Acid+H].

Method B. 50% MeONa in MeOH (14.3 g, 132.3 mmol) was taken into extra MeOH (50 mL) and this mixture was added dropwise with stirring to Intermediate 4 (20 g, 44.1 mmol) in MeOH (300 mL) at 0° C. The reaction mixture was stirred at 0° C. for 1.5 h, suspension filtered and the precipitate was washed with MeOH (ca 50 mL) to afford the crude product. This was recrystallized from acetone (800 mL) and water (650 mL), cooled, and filtered. Resulted solid was washed with acetone (20 mL) to afford the product as a white solid (15.9 g, 72.6%). ¹H NMR (400 MHz, D₂O, ppm): δ 7.97 (s, 1H); 7.55 (d, J=9.2 Hz, 1H); 7.49 (d, J=9.6 Hz, 1H); 7.23 (d, J=14.4 Hz, 1H); 7.06-7.10 (m, 2H); 4.84-4.90 (m, 1H); 4.35 (t, J=8.0 Hz, 2H); 4.02 (t, J=9.0 Hz, 1H); 3.84-3.93 (m, 5H). MS (m/z)): acid form ion 454 [Acid+H].

### Example 3

### Compound of Structure

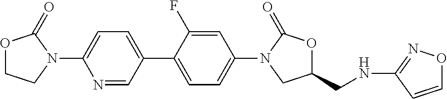

Scheme for Compound of Example 3:

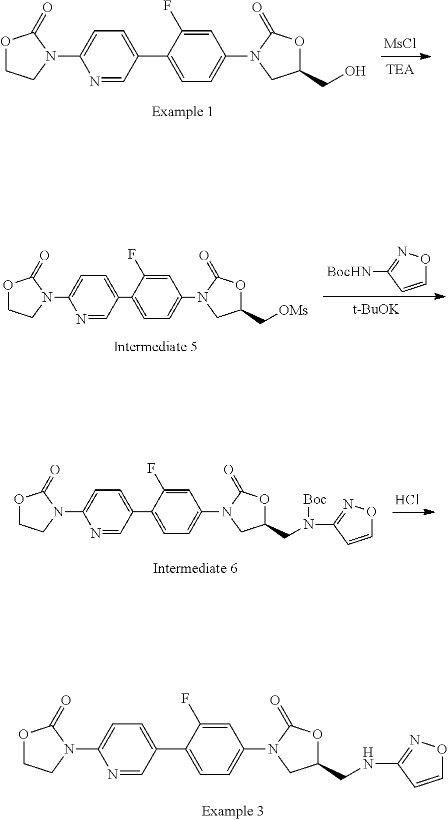

Intermediate 5. Methanesulfonyl chloride (30 μL, 0.46 mmol) was added to a solution of compound of Example 1 (116 mg, 0.30 mmol) and TEA (0.1 mL, 0.93 mmol) in DCM (3 mL) at 0° C. The reaction mixture was stirred at 0° C. for 1 h. Then 5 mL of NH₄Cl (aq) was added. The reaction mixture was extracted with DCM, washed with brine and dried (Na₂SO₄). Evaporation of solvent gave 130 mg of the desired product as a yellow solid.

Intermediate 6. A mixture of tert-butyl isoxazol-3-ylcarbamate (107 mg, 0.58 mmol (prepared as described in the publication US2009/48305) and t-BuOK (71 mg, 0.63 mmol) in DMF (1 mL) was stirred at 0° C. for 2 h. A solution of Intermediate 5 (130 mg, 0.29 mmol) in DMF (1 mL) was added dropwise. The reaction mixture was warmed up to r.t. and stirred o.n. Water (20 mL) was added and the mixture was extracted with EtOAc, washed with brine and dried (Na₂SO₄). After evaporation, the solid was washed with MeOH, and the desired product was obtained as a white solid.

Compound of Example 3. A mixture of Intermediate 6 (150 mg, 0.28 mmol) and conc. HCl (aq) (1 mL) in EtOAc-EtOH (1:1, 3 mL) was stirred at r.t. for 3 h. NaHCO₃ (aq) was added to adjust the pH to ca. 8. The mixture was extracted with DCM, washed with brine and dried (Na₂SO₄). After evaporation, the solid obtained was washed with DCM and Et₂O, and the desired product was collected as a white solid. ¹H NMR: 8.57 (s, 1H); 8.40 (s, 1H); 8.20 (d, J=8.8 Hz, 1H); 8.06 (d, J=8.8 Hz, 1H); 7.67 (s, 1H); 7.64 (dd, J=5.6, 3.2 Hz, 1H); 7.47 (dd, J=8.4, 1.2 Hz, 1H); 6.58 (t, J=6.0 Hz, 1H); 6.02 (d, J=1.2 Hz, 1H); 4.93 (m, 1H); 4.49 (t, J=8.0 Hz, 2H); 4.22 (t, J=8.8 Hz, 3H); 3.88 (dd, J=8.8, 6.4 Hz, 1H); 3.47 (t, J=5.6 Hz, 2H). MS (m/z): 440 [M+H].

### Example 4

### Compound of Structure

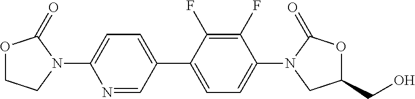

Scheme for Compound of Example 4:

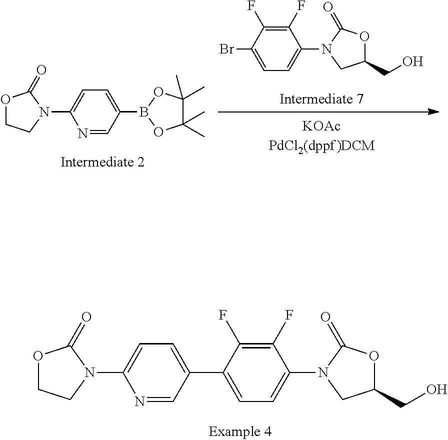

Compound of Example 4. The compound of Example 4 was prepared analogously to the preparation of the Compound of Example 3, using the following reagents: Intermediate 2 (290 mg, 1.0 mmol); Intermediate 7 (308 mg, 1.0 mmol; prepared as described in the publication US2009/48305); Cs₂CO₃ (326 mg, 1.0 mmol) and PdCl₂(dppf)DCM (75 mg, 0.1 mmol). The title compound was obtained as a white solid. ¹H NMR: 8.61 (s, 1H); 8.21 (d, J=8.8 Hz, 1H); 8.11 (d, J=8.8 Hz, 1H); 7.50 (m, 2H); 5.26 (t, J=5.2 Hz, 1H); 4.79 (m, 1H); 4.50 (t, J=8.0 Hz, 2H); 4.22 (t, J=8.0 Hz, 2H); 4.13 (t, J=8.0 Hz, 1H); 3.90 (t, J=8.0 Hz, 1H); 3.70 (m, 1H); 3.61 (m, 1H). MS (m/z): 392 [M+H].

### Example 5

### Compound of Structure

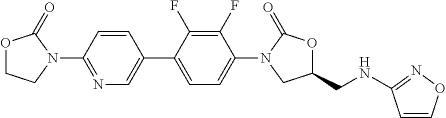

Scheme for Compound of Example 5:

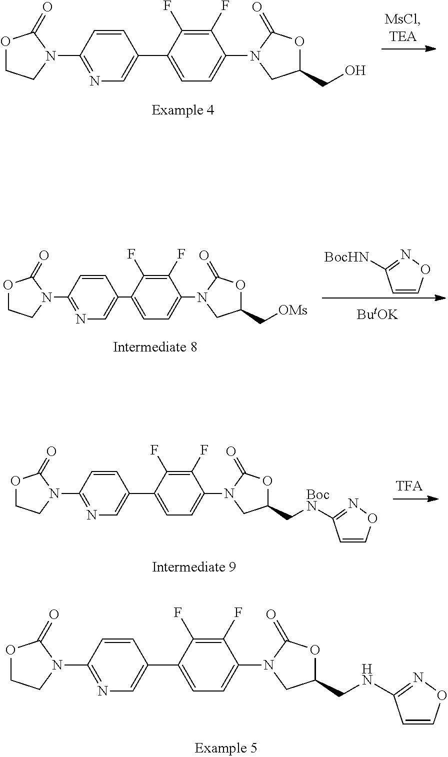

Intermediate 8. Intermediate 8 was prepared analogously to the preparation of the Intermediate 5 as described for the preparation of the Compound of Example 3, using the following reagents: MsCl (68 μL, 1.07 mmol); Compound of Example 4 (278 mg, 0.71 mmol) and triethylamine (307 μL, 2.13 mmol). The desired product was obtained as a yellow solid.

Intermediate 9. Intermediate 9 was prepared analogously to the preparation of the Intermediate 6 as described for the preparation of the Compound of Example 3, using the following reagents: Intermediate 8 (300 mg, 0.64 mmol); tert-butyl isoxazol-3-ylcarbamate (236 mg, 1.28 mmol) and t-BuOK (158 mg, 1.41 mmol). The desired product was obtained as a yellow solid.

Compound of Example 5. Compound of Example 5 was prepared analogously to the preparation of the Compound of Example 3, using the following reagents: TFA (55 μL, 0.72 mmol); Intermediate 9 (100 mg, 0.18 mmol). The desired product was obtained as a white solid. ¹H NMR: 8.61 (s, 1H); 8.40 (d, J=1.6 Hz, 1H); 8.21 (d, J=8.8 Hz, 1H); 8.08 (m, 1H); 7.50 (m, 2H); 6.60 (t, J=6.0 Hz, 1H); 6.02 (d, J=1.6 Hz, 1H); 4.94 (m, 1H); 4.49 (m, 2H); 4.20 (m, 3H); 3.89 (dd, J=9.2, 7.2 Hz, 1H); 3.49 (t, J=5.6 Hz, 2H). MS (m/z): 458 [M+H].

### Example 6

### Compound of Structure

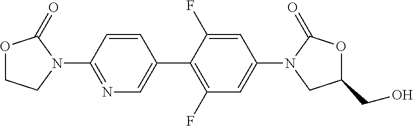

Scheme for Compound of Example 6:

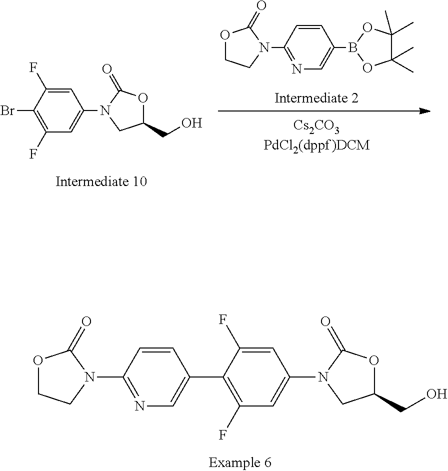

Compound of Example 6. Compound of Example 6 was prepared analogously to the preparation of the Compound of Example 6, using the following reagents: Intermediate 10 (154 mg, 0.5 mmol; prepared analogously to the preparation of Intermediate 3 as described in the publication US2003/13737, except using 3,5-difluorophenyl carbamate derivative instead of 3-fluorophenyl carbamate derivative), Intermediate 2 (145 mg, 0.5 mmol), Cs₂CO₃ (163 mg, 0.5 mmol) and PdCl₂(dppf)DCM (37 mg, 0.05 mmol). The desired compound was obtained as a white solid. ¹H NMR: 8.47 (s, 1H); 8.22 (d, J=8.8 Hz, 1H); 7.95 (d, J=9.6 Hz, 1H); 7.52 (s, 1H); 7.50 (s, 1H); 5.25 (t, J=6.8 Hz, 1H); 4.76-4.80 (m, 1H); 4.49 (t, J=8.0 Hz, 2H); 4.22 (t, J=8.0 Hz, 2H); 4.13 (t, J=9.2, Hz, 1H); 3.88 (dd, J=8.8, 6.0 Hz, 1H); 3.68-3.72 (m, 1H); 3.56-3.59 (m, 1H). MS (m/z): 392 [M+H].

### Example 7

### Compound of Structure

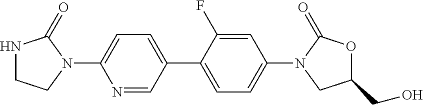

Scheme for Compound of Example 7:

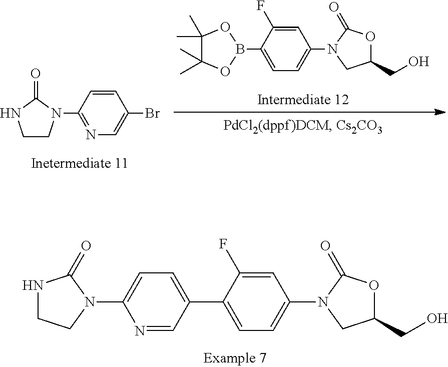

Compound of Example 7. Compound of Example 7 was prepared analogously to the preparation of the Compound of Example 1, except using the following reagents: Intermediate 11 (107.0 mg, 0.489 mmol; prepared analogously to publication US2003/0166620), Intermediate 12 (150.0 mg, 0.445 mmol; prepared analogously to publication PCT WO2009/120789, except using 3-(4-bromo-3-fluorophenyl)-5(R)-(hydroxymethyl)-oxazolidin-2-one instead of 3-(4-bromo-3-fluorophenyl)-5(R)-(tert-butyl-dimethyl-silanyloxymethyl)-oxazolidin-2-one), Cs₂CO₃ (145.0 mg, 0.445 mmol) and PdCl₂(dppf)DCM (30.0 mg). The product was obtained as a white solid. ¹H NMR: 8.48 (s, 1H); 8.28 (d, J=8.8 Hz, 1H); 7.90-7.93 (m, 1H); 7.59-7.67 (m, 2H); 7.46 (dd, J=6.4, 2.0 Hz, 1H); 7.29 (s, 1H); 5.25 (t, J=6.4 Hz, 1H); 4.72-4.78 (m, 1H); 4.14 (t, J=9.2 Hz, 1H); 4.05 (t, J=7.2 Hz, 2H); 3.89 (dd, J=6.4, 2.8 Hz, 1H); 3.68-3.73 (m, 1H); 3.56-3.61 (m, 1H); 3.44 (t, J=8.4 Hz, 2H). MS (m/z): 373 [M+H].

### Example 8

### Compound of Structure

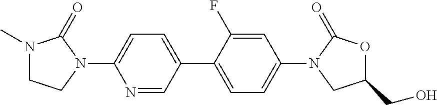

Scheme for Compound of Example 8:

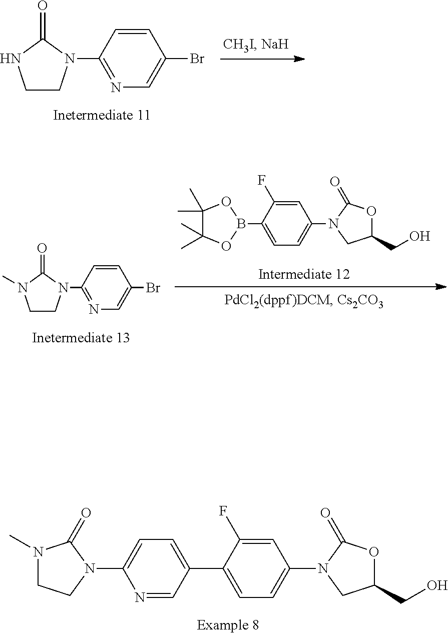

Intermediate 13. Intermediate 11 (102.0 mg, 0.421 mmol) was dissolved in DMF (1 mL) and cooled to 0° C. NaH (80% in mineral oil, 25.3 mg, 0.843 mmol) was added and the reaction mixture was stirred for 30 min. CH₃I (64 μL, 1.26 mmol) was added slowly, and the reaction mixture was warmed up to r.t. and stirred for 2 h. After cooling to 0° C., water (2 mL) was added and the mixture was extracted with EtOAc (10 mL). The EtOAc layer was washed with brine (5 mL×2), dried (Na₂SO₄) and concentrated under vacuum. The resulting solid was rinsed with ether and the desired product was obtained as a white solid. MS (m/z): 256 [M+H].

Compound of Example 8. Compound of Example 8 was prepared analogously to the preparation of the Compound of Example 7, using the following reagents: Intermediate 13 (91.5 mg, 0.357 mmol); Intermediate 12 (110.0 mg, 0.325 mmol); Cs₂CO₃ (106.0 mg, 0.325 mmol) and PdCl₂(dppf)DCM (22.0 mg). The product was obtained as a white solid. ¹H NMR: 8.48 (s, 1H); 8.29 (d, J=8.8 Hz, 1H); 7.91-7.94 (m, 1H); 7.59-7.67 (m, 2H); 7.46 (dd, J=6.8, 2.0 Hz, 1H); 5.25 (t, J=5.2 Hz, 1H); 4.73-4.78 (m, 1H); 4.14 (t, J=9.6 Hz, 1H); 3.98 (t, J=7.2 Hz, 2H); 3.89 (dd, J=6.0, 2.8 Hz, 1H); 3.68-3.73 (m, 1H); 3.56-3.61 (m, 1H); 3.49 (t, J=8.0 Hz, 2H); 2.82 (s, 3H). MS (m/z): 387 [M+H].

### Example 9

### Compound of Structure

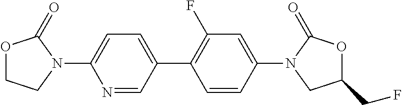

Scheme for Compound of Example 9:

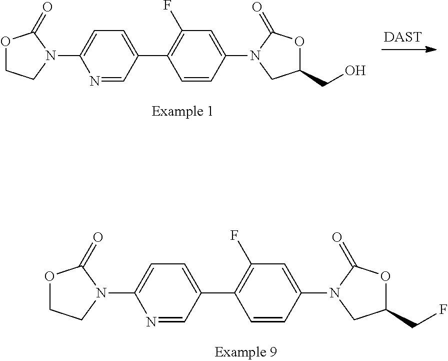

Compound of Example 9. The Compound of Example 1 (65 mg, 0.17 mmol) was dissolved in DCM (2 mL) and cooled to −70° C. DAST (diethylaminosulfur trifluoride) (35 mg, 0.22 mmol) was added to the mixture and the reaction mixture was warmed up to r.t. The reaction was quenched with sat. NH₄Cl and extracted with DCM. The organic layers were combined and washed with brine and dried (Na₂SO₄). Solvent was removed and the residue was purified by preparative TLC (5% MeOH in DCM). The product was obtained as a white solid. ¹H NMR: 8.57 (s, 1H); 8.20 (dd, J=8.8, 0.8 Hz, 1H); 8.04-8.07 (m, 1H); 7.64-7.68 (m, 1H); 7.50 (dd, J=8.4, 2.4 Hz, 1H); 4.96-5.18 (m, 1H); 4.63-4.79 (m, 2H); 4.52 (dd, J=10.8, 8.0 Hz, 2H); 4.20-4.26 (m, 3H); 3.95 (dd, J=9.2, 6.4 Hz, 1H). MS (m/z): 376 [M+H].

### Example 10

### Compound of Structure

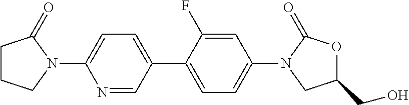

Scheme for Compound of Example 10:

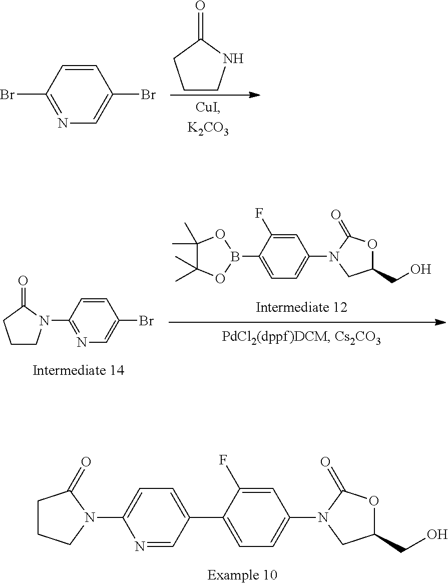

Intermediate 14. Toluene (30 mL) was added to a mixture of pyrrolidin-2-one (3.3 g, 38.0 mmol), 2,5-dibromoprydine (3 g, 12.7 mmol), CuI (0.25 g, 10 mol %), (1S,2S)-(−)-1,2-diaminocyclohexane (0.22 g, 15 mol %) and K₂CO₃ (3.5 g, 25.4 mmol). The mixture was refluxed for 3 h. After cooling to r.t., the mixture was filtered and washed with EtOAc. The combined EtOAc was condensed and the residue was purified by column chromatography (EtOAc: Petroleum ether=1:20 to 1:8). The product was obtained as a white solid.

Compound of Example 10. Compound of Example 10 was prepared analogously to the preparation of the Compound of Example 7, using the following reagents: Intermediate 14 (270 mg, 1.19 mmol); Intermediate 12 (260 mg, 0.77 mmol), Cs₂CO₃ (310 mg, 0.95 mmol) and PdCl₂(dppf) (40 mg). The product was obtained as a white solid. ¹H NMR: 8.58 (s, 1H); 8.42 (d, J=8.8 Hz, 1H); 8.03 (d, J=8.8 Hz, 1H); 7.62-7.69 (m, 2H); 7.49 (dd, J=10.8, 2.0 Hz, 1H); 5.25 (s, 1H); 4.73-4.77 (m, 1H); 4.15 (t, J=9.2 Hz, 1H); 4.04 (t, J=7.6 Hz, 2H); 3.92 (dd, J=8.8, 6.0 Hz, 1H); 3.72 (d, J=11.6 Hz, 1H); 3.60 (t, J=4.8 Hz, 1H); 2.62 (t, J=8.0 Hz, 2H); 2.04-2.12 (m, 2H). MS (m/z): 372 [M+H].

### Example 11

### Compound of Structure

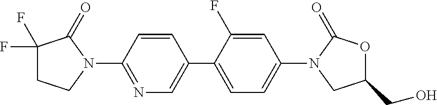

Scheme for Compound of Example 11:

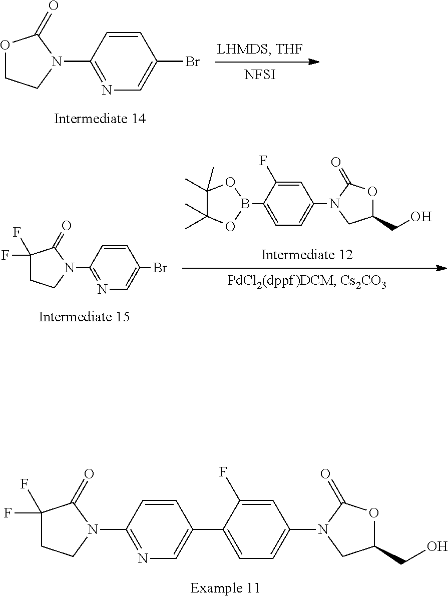

Intermediate 15. LHMDS (9.8 mL, 10.4 mmol) was added to a solution of Intermediate 14 (1.0 g, 4.1 mmol) in THF (20 mL) at −78° C. The mixture was stirred at −78° C. for 45 min, and then N-fluorobenezenesulfonimide (NFSI, 2.9 g, 9.1 mmol) in THF (10 mL) was added dropwise. The mixture was stirred at −78° C. for 2 h, then warmed to −20° C. and stirred for another 0.5 h. EtOAc (400 mL) was added and the mixture was washed with brine and dried (Na₂SO₄). Evaporation of solvent and purification by silica gel column chromatography gave the desired product as a white solid. MS (m/z): 277 [M+H].

Compound of Example 11. Compound of Example 11 was prepared analogously to the preparation of the Compound of Example 7, using the following reagents: Intermediate 15 (75 mg, 0.27 mmol); Intermediate 12 (137 mg, 0.41 mmol); Cs₂CO₃ (88 mg, 0.27 mmol) and PdCl₂(dppf)DCM (22.5 mg, 0.03 mmol). The title compound was obtained as a white solid. ¹H NMR: 8.68 (s, 1H); 8.38 (d, J=8.4 Hz, 1H); 8.14 (d, J=8.8 Hz, 1H); 7.65-7.70 (m, 2H); 7.49 (dd, J=8.4, 2.0 Hz, 1H); 5.26 (t, J=5.6 Hz, 1H); 4.74-4.78 (m, 1H); 4.10-4.17 (m, 3H); 3.89 (dd, J=8.8, 6.0 Hz, 1H); 3.68-3.73 (m, 1H); 3.32-3.61 (m, 1H); 2.70-2.81 (m, 2H). MS (m/z): 408 [M+H].

### Example 12

### Compound of Structure

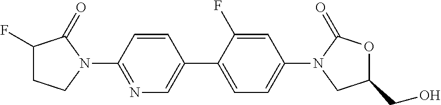

Scheme for Compound of Example 12:

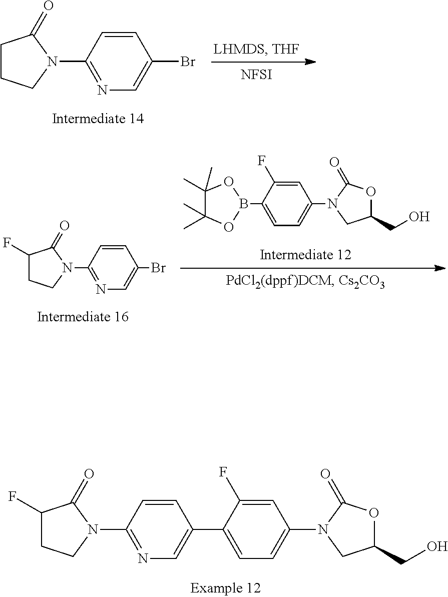

Intermediate 16. Intermediate 16 was prepared analogously to the preparation of the Intermediate 15 as described for the preparation of the Compound of Example 12, using the following reagents: Intermediate 15 (1.0 g, 4.1 mmol); LHMDS (9.8 mL, 10.4 mmol); N-fluorobenezenesulfonimide (NFSI, 2.9 g, 9.1 mmol). The product was obtained as a white solid. MS (m/z): 259 [M+H].

Compound of Example 12. Compound of Example 13 was prepared analogously to the preparation of the Compound of Example 7, using the following reagents: Intermediate 16 (130 mg, 0.5 mmol); Intermediate 12 (168 mg, 0.5 mmol); Cs₂CO₃ (163 mg, 0.5 mmol) and PdCl₂(dppf)DCM (37 mg, 0.05 mmol). The title compound was obtained as a white solid. ¹H NMR: 8.63 (s, 1H); 8.42 (d, J=8.8 Hz, 1H); 8.10 (d, J=9.2 Hz, 1H); 7.68 (t, J=8.8 Hz, 2H); 7.48 (dd, J=8.8, 2.4 Hz, 1H); 5.45, 5.40 (t, J=8.0 Hz, 1H); 5.26 (t, J=5.6 Hz, 1H); 4.74-4.78 (m, 1H); 4.17 (dd, J=21.6, 11.2 Hz, 2H); 3.85-3.91 (m, 2H); 3.68-3.73 (m, 1H); 3.38-3.61 (m, 1H); 2.63-2.68 (m, 1H); 2.16-2.26 (m, 1H). MS (m/z): 390 [M+H].

### Example 13

### Compound of Structure

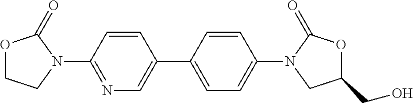

Scheme for Compound of Example 13:

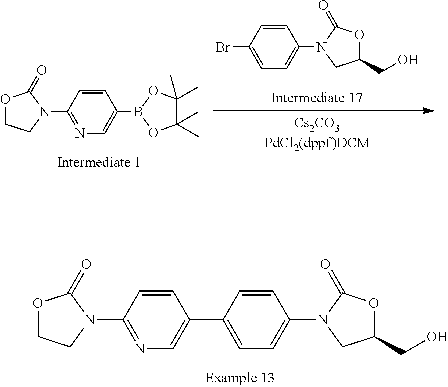

Compound of Example 13. Compound of Example 13 was prepared analogously to the preparation of the Compound of Example 1, using the following reagents: Intermediate 17 (95 mg, 0.35 mmol, prepared analogously to the preparation of Intermediate 3 per publication US2003/13737, except using des-fluorophenyl carbamate derivative instead of 4-bromo-3-fluorophenyl carbamate derivative), Intermediate 1 (102 mg, 0.35 mmol), Cs₂CO₃ (114 mg, 0.35 mmol) and PdCl₂(dppf)DCM (13 mg, 0.018 mmol). The product was obtained as a white solid. ¹H NMR: 8.71 (s, 1H); 8.16 (s, 2H); 7.76 (d, J=8.4 Hz 2H); 7.68 (d, J=8.8 Hz 2H); 5.26 (br, 1H); 4.73 (m, 1H); 4.49 (t, J=8.0 Hz, 2H); 4.22 (t, J=8.0 Hz, 2H); 4.14 (t, J=9.2 Hz, 1H); 3.88 (dd, J=8.8, 6.4 Hz, 1H); 3.71 (dd, J=12.0, 2.8 Hz, 1H); 3.57 (dd, J=12.0, 3.6 Hz, 1H). MS (m/z): 356 [M+H].

### Reference Materials

Previously described Reference Compound 14 and Reference Compound 15 (hereafter also referred to as Ref. Cpd. 14 and Ref. Cpd. 15, respectively) were synthesized by modifying methods and reagents used to prepare compounds as described herein.

Reference Compound 14: N-[[(5S)-3-[3-fluoro-4-[6-(2-oxooxazolidin-3-yl)-3-pyridyl]phenyl]-2-oxo-oxazolidin-5-yl]methyl]acetamide (Example 143 of publication US 2003/0166620). ¹H NMR: 8.56 (s, 1H); 8.28 (t, J=6.0 Hz, 1H); 8.17 (d, J=8.8 Hz, 1H); 8.06 (d, J=8.8 Hz, 1H); 7.65 (dd, J=16.4, 4.0 Hz, 2H); 7.43 (dd, J=8.4, 2.0 Hz, 1H); 4.76-4.79 (m, 1H); 4.49 (t, J=8.0 Hz, 2H); 4.16-4.24 (m, 3H); 3.79 (dd, J=8.8, 6.4 Hz, 1H); 3.44 (t, J=5.2 Hz, 2H); 1.85 (s, 3H). MS (m/z): 415 [M+H].

Reference Compound 15: (5R)-3-[3-fluoro-4-[6-(2-methyltetrazol-5-yl)-3-pyridyl]phenyl]-5-(hydroxymethyl)oxazolidin-2-one (TR-700 or torezolid; reference publications PCT WO2005/058886 and PCT WO2010/042887). ¹H NMR: 8.20-8.25 (m, 2H); 7.70-7.79 (m, 2H); 7.55 (dd, J=8.8, 2.0 Hz, 1H); 5.26 (t, J=5.2 Hz, 1H); 4.75-4.79 (m, 1H); 4.48 (s, 3H); 4.17 (t, J=8.8 Hz, 1H); 3.92 (dd, J=8.8, 6.0 Hz, 1H); 3.70-3.73 (m, 1H); 3.58-3.62 (m, 1H). MS (m/z): 371 [M+H].

### Utility and Testing

Compounds of the subject invention exhibit potent activities against a variety of microorganisms, including Gram-positive microorganisms. Accordingly, compounds of the subject invention have useful antibacterial activity. Thus, compounds of the present invention are useful antimicrobial agents and may be effective against a number of human and veterinary pathogens, including gram positive aerobic bacteria such as multiply-resistant Staphylococci, Enterococci, and Streptococci, as well as anaerobic microorganisms such as Bacteroides and Clostridia species, and acid-fast microorganisms such as Mycobacterium tuberculosis and Mycobacterium avium. Importantly, certain compounds of this invention possess activity against linezolid-susceptible Gram-positive infections (including MRSA, VRE, and PRSP), against linezolid-resistant infections (including resistant strains of S. aureus and Enterococci), as well as against fastidious Gram-negative pathogens implicated in respiratory tract infections and meningitis (including H. influenzae and M. catarrhalis). This beneficially broad spectrum of activity beneficially coupled with attenuated propensity for myelosuppression is unprecedented among oxazolidinone antibacterials.

Compounds of this invention can have useful activity against a variety of pathogenic microorganisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA. Minimum inhibitory concentration (MIC) refers to the lowest concentration of drug (μg/mL) that inhibits visible growth of the organism. Lower MIC values indicate a higher antibacterial activity. Typically, compounds of the present invention have useful potency against Gram-positive or Gram-negative pathogens with MIC values of ≦8 μg/mL. To measure useful antibacterial activity against multiple bacterial isolates or strains, MIC₉₀ (or minimal inhibitory concentration of the drug that inhibits visible growth of 90% of the microbes in each bacterial specie panel) is determined. Lower MIC number indicates an elevated degree of useful antibacterial activity, while higher MIC number indicates a reduced antibacterial activity. An agent with too high MIC against a bacterial pathogen can not be used for the treatment of infections caused by such microbial species.

Thus, MIC serves to define the potential utility of antibacterial agent for anti-infective therapy in general and specific indication(s) thereof. For example, oxazolidinone drug linezolid generally displays MIC in the range of 2-4 μg/mL against Staphylococci species (such as S. aureus), and has subsequently been approved for the treatment of infections caused by these pathogens. In contrast, linezolid is not indicated for therapy of infections caused by fastidious Gram-negative pathogen H. influenzae for which a MIC₉₀ of 16 mg/mL is reported. Likewise, linezolid is not indicated for treatment of diseases caused by linezolid-resistant bacterial strains, against which it displays MICs of 8 μg/mL and higher, since the drug may not be effective against such infections. Several cases of the linezolid therapy failure when encountering such resistant bacteria have been reported, for example, by Garcia et al. in J. Amer. Med. Association (JAMA), 2010, vol. 303, No. 22, p. 2260.

The useful activity of compounds of the present invention against an expanded set of clinical isolates of Staphylococcus aureus (including methicillin-resistant strain, MRSA), Enterococci (including Enterococcus faecalis and Enterococus faecium), and Staphylococcus pneumonia is illustrated by the MIC₉₀ data in Table 1.

TABLE 1
Antibacterial activity against linezolid-susceptible pathogens.

| | S. aureus | Enterococci | S. pneumoniae |
|---|---|---|---|
| | (34 strains) | (34 strains) | (34 strains) |
| EXAMPLES | MIC₉₀ μg/mL | MIC₉₀ μg/mL | MIC₉₀ μg/mL |
| Linezolid | 2.0 | 2 | 1 |
| Example 1 | 0.125 | 0.25 | 0.06 |
| Ref. Cpd. 15* | 0.5 | 0.5 | 0.25 |

*TR-700, torezolid; reference publication PCT WO2005/058886.

As clear from MIC₉₀ for linezolid and the representative compound of this invention of Example 1, the numerical values for the latter antibacterial compound are about 16-fold, 8-fold, and 16-fold fold lower than corresponding MIC₉₀ values for linezolid, against S. aureus, Enterococci species, and S. pneumoniae, respectively. As illustrated by MIC₉₀ for the Reference Compound 15 (torezolid; ref. publications PCT WO2005/058886 and PCT WO2010/042887) and the compound of Example 1, the numerical values for the latter antibacterial agent of this invention are about 4-fold, 2-fold, and 4-fold fold lower than corresponding MIC₉₀ values for the Reference Compound 15, against S. aureus, Enterococci species, and S. pneumoniae, respectively.

This dramatically elevated antibacterial potency would not have been anticipated since the structures for compound of Example 1 and for the Reference Compound 15 both feature four identical key fragments: pyridyl, fluorophenyl and oxazolidinone rings, and 5-hydroxymethyl group. Thus, aforementioned two compounds would have been expected to exhibit generally similar levels of the antibacterial activity, rather than a surprising superiority for the novel compound of Example 1.

Likewise, additional compounds of this invention (such as compounds of Examples 7, 10, and 13) demonstrate lower range MIC values in these tests, as compared to linezolid or the Reference Compound 15, torezolid. These data illustrate a surprisingly enhanced potency for the compounds of this invention against key Gram-positive pathogens.

The useful activity of representative compounds of the present invention against several isolates of linezolid-resistant Staphylococcus aureus, Enterococus faecalis and Enterococcu faecium illustrated by the MIC data of Table 2.

TABLE 2
Antibacterial activity against linezolid-resistant strains.

| | S. aureus | E. faecalis | E. faecium |
|---|---|---|---|
| | (no. of strains) | (no. of strains) | (no. of strains) |
| EXAMPLES | MIC, μg/mL | MIC, μg/mL | MIC, μg/mL |
| Linezolid | 16 (2 strains) | 16 (4 strains) | 16 (5 strains) |
| | >16 (3 strains) | | |
| Ref. Cpd. 15* | 4 (2 strains) | 4 (4 strains) | 2 (1 strain) |
| | 8 (3 strains) | | 4 (3 strains) |
| Example 1 | 0.5 (1 strain) | 0.5 (1 strain) | 0.5 (5 strains) |
| | 1 (1 strain) | 1 (3 strains) | |
| | 2 (3 strains) | | |
| Example 7 | 1 (2 strains) | 1 (1 strain) | 0.5 (1 strain) |
| | 4 (3 strains) | 2 (1 strains) | 1 (4 strains) |
| | | 4 (2 strains) | |
| Example 10 | 1 (2 strains) | 1 (1 strain) | 1 (5 strains) |
| | 4 (3 strains) | 2 (3 strains) | |

*TR-700, torezolid; reference publication PCT WO2005/058886.

As illustrated by the data of Table 2, the numerical MIC values for activity of the representative compounds of this invention against linezolid-resistant strains of S. aureus, E. faecalis and E. faecium are generally at 4 μg/mL or below, or within therapeutically useful MIC values as determined for the drug linezolid against linezolid-sensitive strains (in contrast to elevated linezolid MICs against resistant bacteria illustrated in Table 2, against which the linezolid therapy is not indicated). MIC numbers for the compound of this invention of Example 1 are about 2- to 8-fold, 4- to 8-fold, and 4- to 8-fold lower than respective values for the Reference Compound 15, against linezolid-resistant S. aureus, E. faecalis and E. faecium, respectively. MIC numbers for the compound of this invention of Example 7 are generally up to 2-4-fold lower than respective values for the Reference Compound 15, against clinical isolates of linezolid-resistant S. aureus, E. faecalis and E. faecium, respectively. MIC numbers for the compound of this invention of Example 10 are generally 2-4-fold lower than respective values for the Reference Compound 15, against linezolid-resistant strains of S. aureus, E. faecalis and E. faecium, respectively. These data illustrate a surprisingly enhanced potency for the representative compounds of this invention against Gram-positive linezolid-resistant strains.

This dramatically enhanced antibacterial potency is particularly surprising for the compound of Example 1, since the structures for this compound and for the Reference Compound 15 both feature four identical elements: pyridyl, fluorophenyl and oxazolidinone rings, and the right-side hydroxymethyl side chain. Thus, the two structures would have been expected to exhibit only similar levels of the antibacterial activity, rather than a surprising superiority observed for the novel compound of Example 1.

The useful activity of representative compounds of the present invention against several isolates of H. influenzae is illustrated by the MIC data of Table 3. H. influenzae is a fastidious Gram-negative pathogen implicated in multiple infections, including pneumonia and bacterial meningitis. No oxazolidinone agent is presently approved for the treatment of H. influenzae infections.

TABLE 3
Antibacterial activity against H. infulenzae.

| | H. influenzae |
|---|---|
| | (no. of strains) |
| EXAMPLES | MIC, μg/mL |
| Linezolid | 8 (3 strains) |
| | 16 (2 strains) |
| Ref. Cpd. 15* | 8 (4 strains) |
| | >16 (1 strain) |
| Example 1 | 2 (4 strains) |
| | 4 (1 strain) |
| Example 10 | 4 (5 strains) |
| Example 13 | 4 (2 strains) |

*TR-700, torezolid; ref. PCT WO2005/058886.

The representative data of Table 3 are in agreement with reported for linezolid MIC₉₀ of 16 mg/mL vs. H. influenzae species, against which the drug is not indicated due to the low activity. Likewise, the Reference Compound 15 displays MIC values in the range of 8 to >16 mg/mL. In contrast, MIC values for the representative compounds of this invention of Example 1 and of Example 10 are generally at 4 mg/mL or below. MIC values for the compounds of this invention of Example 1, of Example 10, and of Example 13 against H. influenzae are generally 2- to 4-fold lower than respective values for the Reference Compound 15, torezolid. These data illustrate a surprisingly enhanced potency for the representative compounds of this invention against H. influenzae species. Thus, in contrast to the Reference Compound 15, compounds of this invention offer therapeutic coverage against H. influenzae not provided by said comparator.

This dramatically increased activity against H. influenzae species is particularly surprising for the compound of Example 1 that features key elements also present in the Reference Compound 15 (pyridyl, fluorophenyl and oxazolidinone rings, and the hydroxymethyl side chain). Thus, only similar antibacterial activity vs. H. influenzae would have been ordinarily anticipated for these two compounds.

MIC is the critical parameter for antibacterial agents, as this value serves to define the concentration and exposure needed for an effective therapy, as well as the drug dose and administration frequency needed to achieve the same. Thus, a 2-fold difference in MIC can translate into a 2-fold reduced amount (or dose) of the drug agent needed for a successful therapy, or may allow for less frequent (for example, once-daily) dosing as compared to a similar but less potent agent (i.e. with higher MIC) that would require more frequent dosing. Additionally, a lower MIC can translate into a reduced frequency of adverse effects for more potent drug with otherwise similar properties, since the incidence and severity of adverse effects generally correlates with the dose, and the latter could be reduced as compared to a less potent drug with higher MICs.

In addition to the desired levels of antibacterial activity, an effective antibacterial agent needs to be safe and well tolerated in the course of the therapy. It is well recognized that the utility of oxazolidinone antibacterials is limited due to the potential for serious adverse effects. Among these, myelosuppression or bone marrow toxicity is the chief factor limiting utility of the only approved oxazolidinone linezolid (Zyvox®), as reflected in the warning included with the drug's prescribing information. The latter type of the oxazolidinone toxicity manifested in a bone marrow suppression (also referred to as hematopoietic toxicity or myelosuppression) was reported, for example, by Monson et al. in Clinical Infectious Diseases, 2002, vol. 35, pp. e29-31. This toxicity is manifested in such adverse effects for Zyvox® as anemia, leukopenia, pancytopenia, and thrombocytopenia.

The potential of representative compounds of this invention for myelosuppression has been evaluated in a 5-days mouse sub-acute toxicity test. For comparison purposes, Reference Compound 14 (Example 143 of publication US 2003/0166620) and Reference Compound 15 (TR-700 or torezolid; publications PCT WO2005/058886 and PCT WO2010/042887) have been included. The compounds were dosed to mice at 37.5 mg/kg/day on a twice-daily schedule. On day 5, blood was harvested for hematology testing. The toxicity was evaluated based on the reduction in the number of reticulocytes, previously described as the key marker for myelosuppression signs in pre-clinical testing of oxazolidinones, including linezolid. Thus, a higher degree of a reduction in the reticulocyte count is indicative of an elevated toxicity, while a lower drop in the reticulocyte count indicates a reduced propensity to the myelosuppression effect on the blood cells.

Representative reticulocyte count data are summarized in the Table 5 below.

TABLE 5
Reticulocyte count changes in 5-days sub-acute mouse model.

| | | Ratio for |
|---|---|---|
| | | reticulocyte count |
| | Reticulocyte | relative to that |
| | count on Day 5, | for compound of |
| EXAMPLES | cell count, ×10⁹/L | Example 1 |
| Example 1 | 127.5 | 1 |
| Ref. Cpd. 14* | 50.0 | 0.39 |
| Ref. Cpd. 15** | 19.1 | 0.15 |

*Reference compound of the publication US 2003/0166620.
**TR-700, torezolid; ref. PCT WO2005/058886.

As can be seen from the data in Table 5, Reference Compound 14 and Reference Compound 15 (torezolid) induce around a 2.6-fold drop in the reticulocyte cells counts compared to the compound of this invention of Example 1. Thus, the data indicate a surprisingly reduced potential for bone marrow toxicity for the compound of Example 1 as compared to the Reference Compounds 14 and 15.

Above data are particularly surprising since the compound of Example 1 features four key elements also present in the Reference Compound 15 (pyridyl, fluorophenyl, oxazolidinone, and hydroxymethyl groups), and four structural elements present in the Reference Compound 14 (pyridyl, fluorophenyl, and two oxazolidinone rings). Thus, the compound of Example 1 would have been anticipated to possess generally similar to the Reference Compounds 14 and 15 bone marrow toxicity. In a contrast, the compound of Example 1 offers a dramatically improved safety profile essential for a successful therapy with minimal side effects due to myelosuppression.

Any drug is evaluated on the balance of its useful activity vs. potential for toxicity, as described, for example, by Barrett in Current Protocols in Pharmacology, 2005, 13A.1.1-13A.1.8. The pharmacological profile for compounds of this invention can be evaluated by analyzing the toxicity data (of Table 5) in a context of useful activity thereof as represented by MIC₉₀ data (of Table 1). Thus, for the compound of this invention of Example 1 and the comparator Reference Compound 15 (torezolid), dividing the reticulocyte count (of Table 5) by the corresponding MIC₉₀ against S. aureus (of Table 1) gives ratios of 1020 (127.5/0.125=1020) and 76.4 (19.1/0.25=76.4). The compound of Example 1 clearly displays a dramatic and unexpected improvement over the comparator Reference Compound 15 with around a 13-fold difference between the two. These data evidence the surprisingly improved therapeutic potential for compounds of this invention as compared to previously described oxazolidinone agents, including those that feature certain common groups also present in the new compounds (as illustrated above for the comparison of the compound of Example 1 and Reference Compounds 14 and 15).

The data illustrated by Tables 1-5 demonstrate that the representative compounds of the present invention display a unique combination of useful pharmacological properties, including exceptional activity against key Gram-positive pathogens, expanded antibacterial spectrum that covers Gram-positive linezolid-resistant bacteria, as well as fastidious Gram-negative species, and beneficially reduced propensity for adverse effects due to myelosuppression.

Theses data support the utility for the compounds of this invention for anti-infective therapy for diseases caused by both linezolid-sensitive and linezolid-resistant pathogens (including MRSA), as well as those caused by fastidious Gram-negative pathogens (including H. influenzae).

### Administration and Pharmaceutical Formulations

In general, the compounds of the subject invention can be administered in a therapeutically effective amount by any of the accepted modes of administration for agents that serve similar utilities. By way of example, compounds of the subject invention may be administered orally, parenterally, transdermally, topically, rectally, or intranasally. The actual amount of a compound of the subject invention, i.e., the active ingredient, will depend on a number of factors, such as the severity of the disease, i.e., the infection, to be treated, the age and relative health of the subject, the potency of the compound used, the route and form of administration, and other factors, all of which are within the purview of the attending clinician.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices, that is, an ED₅₀ that is much lower than the LD₅₀ are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

When employed as pharmaceuticals, the compounds of the subject invention are usually administered in the form of pharmaceutical compositions. These compounds can be administered by a variety of routes including oral, parenteral, transdermal, topical, rectal, and intranasal.

Compounds provided herein are effective as injectable, oral, inhaleable, or topical compositions. Such compositions are prepared in a manner well known in the pharmaceutical art and comprise at least one active compound.

This invention also includes pharmaceutical compositions which contain, as the active ingredient, one or more of the compounds of the subject invention above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The quantity of active component, that is the compound according to the subject invention, in the pharmaceutical composition and unit dosage form thereof may be varied or adjusted widely depending upon the particular application, the potency of the particular compound and the desired concentration.

The compositions are preferably formulated in a unit dosage form, each dosage containing from about 0.1 to about 2000 mg, more usually about 1 to about 900 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of the subject invention above is employed at no more than about 20 weight percent of the pharmaceutical composition, more preferably no more than about 15 weight percent, with the balance being pharmaceutically inert carrier(s).

An active compound is effective over a wide dosage range and is generally administered in a pharmaceutically or therapeutically effective amount. It, will be understood, however, that the amount of the compound actually administered can be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, compounds or pharmaceutical compositions thereof can be administered orally, topically, transdermally, and/or parenterally at a dosage to obtain and maintain a concentration, that is, an amount, or blood-level of active component in the animal undergoing treatment which will be antibacterially effective. Generally, a therapeutically effective dose of active component will be in the range of about 0.1 mg/kg to about 250 mg/kg, more preferably about 1.0 mg/kg to about 50 mg/kg of body weight/day.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described above. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

The following formulation examples illustrate representative pharmaceutical compositions of the present invention. Amount of a compound of present invention in a formulation composition can be in a range of 10-10000 mg. Preferably, said amount can be in a range of 20-900 mg. More preferably, said amount can be in a range of 50-750 mg, or even more preferably, in a range of 200-600 mg.

### Formulation Example 1

Hard gelatin capsules containing the following ingredients are prepared:

| | Quantity |
|---|---|
| Ingredient | (mg/capsule) |
| Active Ingredient | 200-600 |
| Starch | 100-300 |
| Magnesium stearate | 5-15 |

The above ingredients are mixed and filled into hard gelatin capsules for oral administration.

### Formulation Example 2

A tablet formula is prepared using the ingredients below:

| | Quantity |
|---|---|
| Ingredient | (mg/tablet) |
| Active Ingredient | 50-750 |
| Cellulose, microcrystalline | 100-250 |
| Colloidal silicon dioxide | 10-20 |
| Stearic acid | 5-10 |

The components are blended and compressed to form tablets for oral administration.

### Formulation Example 3

A dry powder inhaler formulation is prepared containing the following components:

| Ingredient | Weight % |
|---|---|
| Active Ingredient | 100-600 |
| Lactose | 40-100 |

The active ingredient is mixed with the lactose and the mixture is added to a dry powder inhaling appliance.

### Formulation Example 4

Tablets, each containing 200-600 mg of active ingredient, are prepared as follows

| | Quantity | |
|---|---|---|
| Ingredient | (mg/tablet) | |
| Active Ingredient | 200-600 | mg |
| Starch | 15-45 | mg |
| Microcrystalline cellulose | 10-35 | mg |
| Polyvinylpyrrolidone | 5-10 | mg |
| (as 10% solution in sterile water) | | |
| Sodium carboxymethyl starch | 5-10 | mg |
| Magnesium stearate | 0.5-2 | mg |
| Talc | 1.0-5 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets for oral administration.

### Formulation Example 5

Capsules, each containing 200-600 mg of medicament are made as follows:

| | Quantity | |
|---|---|---|
| Ingredient | (mg/capsule) | |
| Active Ingredient | 200-600 | mg |
| Starch | 75-150 | mg |
| Magnesium stearate | 1-4 | mg |

The active ingredient, starch and magnesium stearate are blended, passed through a No. 20 mesh U.S. sieve, and filled into hard gelatin capsules for oral administration.

### Formulation Example 6

Suppositories, each containing 200-600 mg of active ingredient are made as follows:

| Ingredient | Amount | |
|---|---|---|
| Active Ingredient | 200-600 | mg |
| Saturated fatty acid glycerides to | 1000-2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 200-600 mg of medicament per 7 mL dose are made as follows:

| Ingredient | Amount | |
|---|---|---|
| Active Ingredient | 200-600 | mg |
| Xanthan gum | 2-8 | mg |
| Sodium carboxymethyl cellulose (11%) | 20-50 | mg |
| Microcrystalline cellulose (89%) | | |
| Sucrose | 1.0-1.75 | g |
| Sodium benzoate | 10-20 | mg |
| Flavor and Color | q.v. | |
| Purified water to | 5-7 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| | Quantity | |
|---|---|---|
| Ingredient | (mg/capsule) | |
| Active Ingredient | 200-600 | mg |
| Starch | 200-410 | mg |
| Magnesium stearate | 3-6 | mg |

The active ingredient, starch, and magnesium stearate are blended, passed through a No. 20 mesh U.S. sieve, and filled into hard gelatin capsules for oral administration.

### Formulation Example 9

A subcutaneous formulation may be prepared as follows:

| Ingredient | Quantity | |
|---|---|---|
| Active Ingredient | 200-600 | mg |
| Corn Oil | 1.0-1.5 | mL |

### Formulation Example 10

A topical formulation may be prepared as follows:

| Ingredient | Quantity | |
|---|---|---|
| Active Ingredient | 1-10 | g |
| Emulsifying Wax | 20-30 | g |
| Liquid Paraffin | 10-20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions that can transiently open the blood-brain barrier.

Other suitable formulations for use in the present invention can be found in Remington's Pharmaceutical Sciences, Mace Publishing Company, Philadelphia, Pa., 17th ed. (1985).

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

The compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While the present invention has been described herein in conjunction with a preferred aspect, a person with ordinary skills in the art, after reading the foregoing specification, can affect changes, substitutions of equivalents and other types of alterations to the invention as set forth herein. Each aspect described above can also have included or incorporated therewith such variations or aspects as disclosed in regard to any or all of the other aspects. The present invention is also not to be limited in terms of the particular aspects described herein, which are intended as single illustrations of individual aspects of the invention. Many modifications and variations of this invention can be made without departing from its spirit and scope, as will be apparent to those skilled in the art. Functionally equivalent methods within the scope of this invention, in addition to those enumerated herein, will be apparent to those skilled in the art from the foregoing descriptions. It is to be understood that this invention is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

What is claimed is:

1. A compound according to formula I 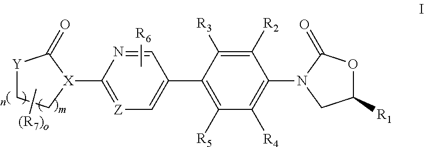
or a pharmaceutically acceptable salt, prodrug, solvate, or hydrate thereof wherein:
R¹ is CH₂OH, CH₂OPO₃H₂, CH₂F, CH₂NHC(═O)OC₁₋₅alkyl, (4-R⁸-1,2,3-triazol-1-yl)methyl, (5-R⁸-isoxazol-3-yl)aminomethyl, or (5-R⁸-isoxazol-3-yl)oxymethyl, wherein R⁸ is H, C₁₋₆alkyl, halo, or CN;
R² and R⁴ are independently H or F;
R³ and R⁵ are independently H, F, CN, or CH₃;
R⁶ is H, halo, or C₁₋₆alkyl;
R⁷ is a single or multiple substituent(s) selected from H, F, C₁₋₆alkyl, or C₃₋₆ cycloalkyl;
X is N, CH, or CF;
Y is NH, NC₁₋₄alkyl, O, CH₂, CHF, or CF₂;
Z is CH, CF, or N;
m, n, and o are independently 0, 1, or 2.

2. The compound of claim 1 wherein
R₁ is CH₂OH, CH₂OPO₃H₂, CH₂F, CH₂NHC(═O)OC₁₋₅alkyl, (4-R⁸-1,2,3-triazol-1-yl)methyl, (5-R⁸-isoxazol-3-yl)aminomethyl, or (5-R⁸-isoxazol-3-yl)oxymethyl, wherein R⁸ is H, C₁₋₆alkyl, halo, or CN; with a proviso that when X is N, and Y is O; then
R⁷ is other than F or C₁₋₆alkyl.

3. The compound of claim 1 with a proviso that when
R₁ is (4-R⁸-1,2,3-triazol-1-yl)methyl, (5-R⁸-isoxazol-3-yl)aminomethyl, or (5-R⁸-isoxazol-3-yl)oxymethyl, then
at least one of R² and R⁴ is F.

4. The compound of claim 1 with a proviso that when
R₁ is CH₂OH, CH₂OPO₃H₂, CH₂F, (4-R⁸-1,2,3-triazol-1-yl)methyl, (5-R⁸-isoxazol-3-yl)aminomethyl, or (5-R⁸-isoxazol-3-yl)oxymethyl; X is N; and Y is O; then
R⁷ is H, or o is 0.

5. The compound of claim 1 or salt thereof, wherein R¹ is CH₂OH or CH₂OPO₃H₂; and R⁷ is H or F.

6. The compound of claim 1, wherein R¹ is CH₂OH or CH₂OPO₃H₂, X is N; Y is CH₂, CHF, CF₂, or O; and R⁷ is H.

7. The compound of claim 1, wherein R² and R⁴ are H; and R³ and R⁵ are independently selected from H and F.

8. The compound of claim 1 wherein R¹ is CH₂OH or CH₂OPO₃H₂; m and n are both 1; and o is 0.

9. The compound of claim 1 selected from:

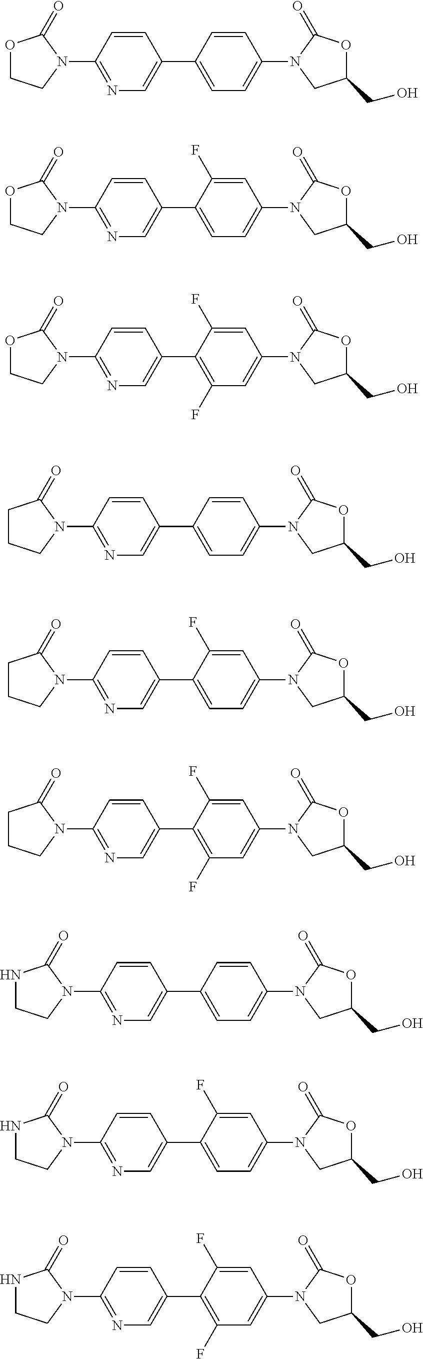

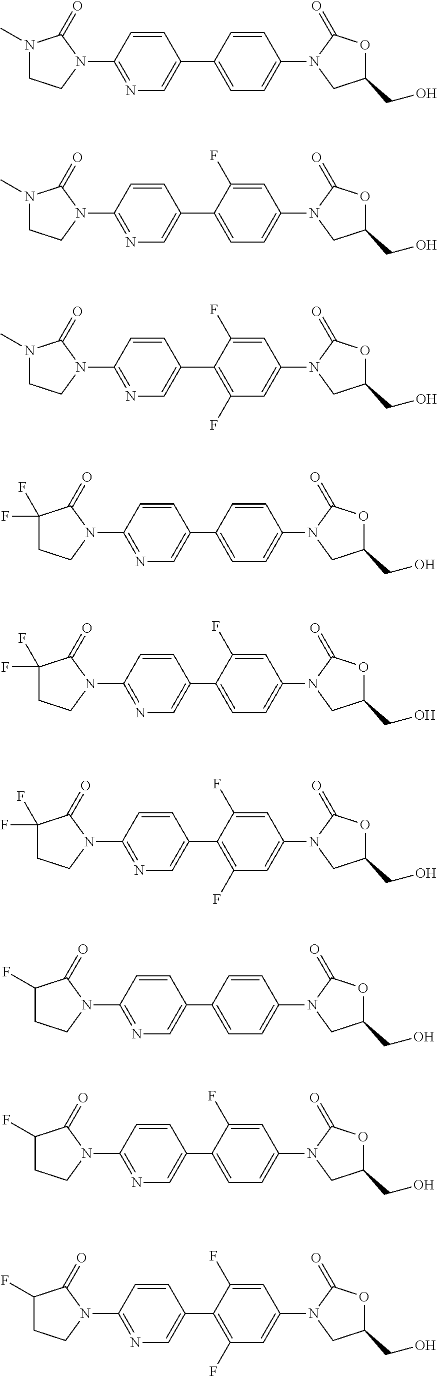

10. The compound of claim 1 selected from:

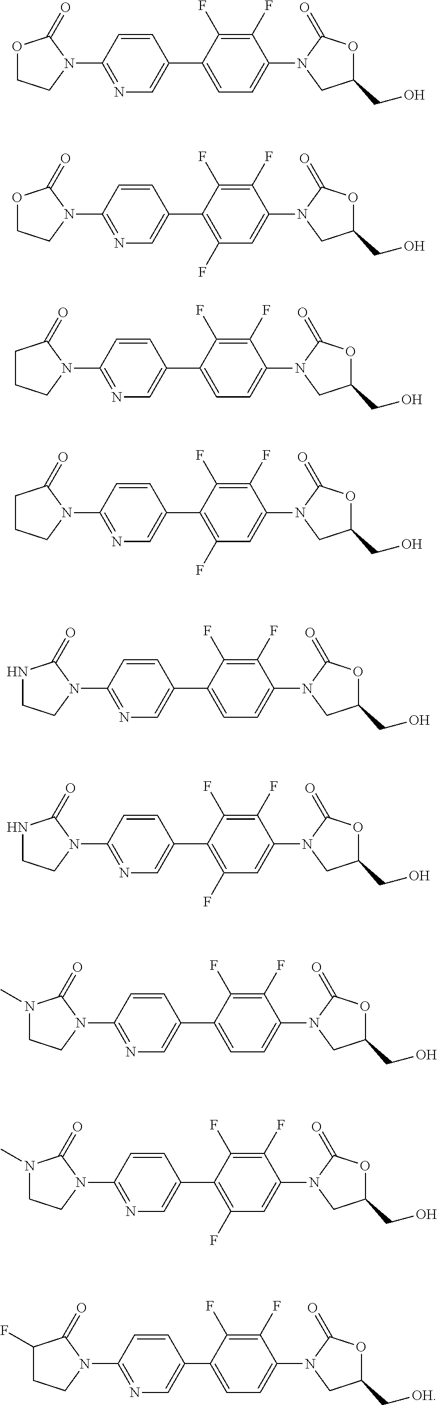

11. The compound of claim 1 selected from:

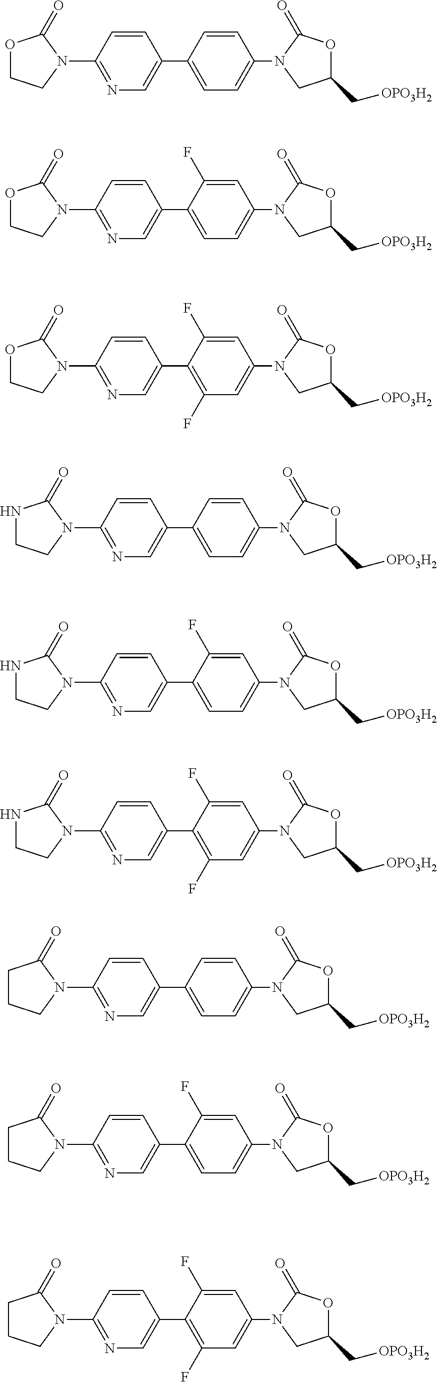

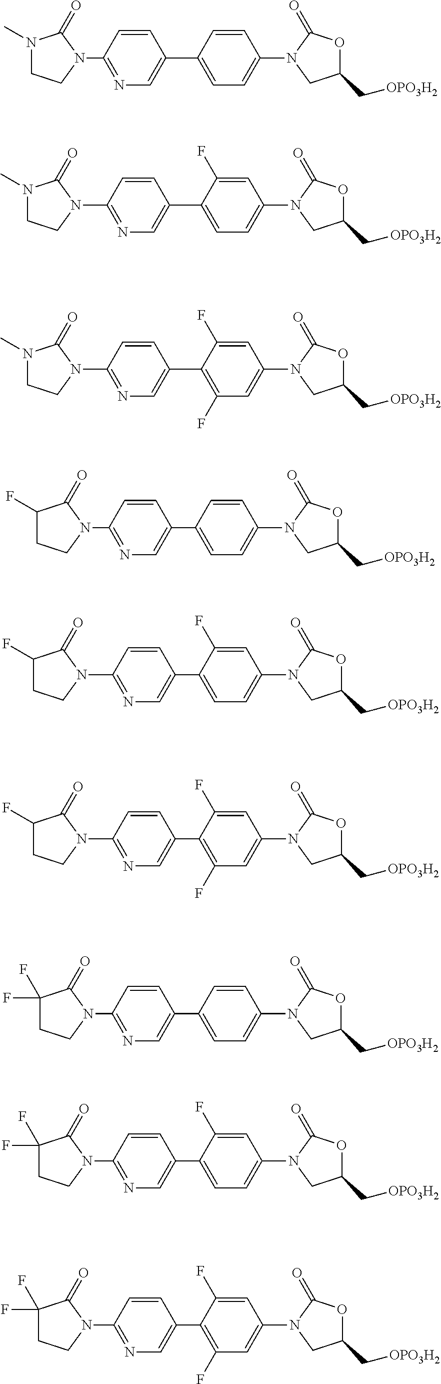

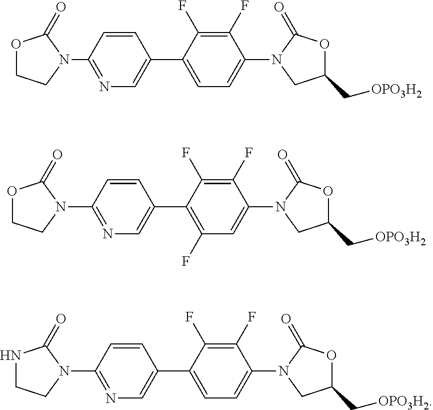

12. The compound of claim 1 selected from:

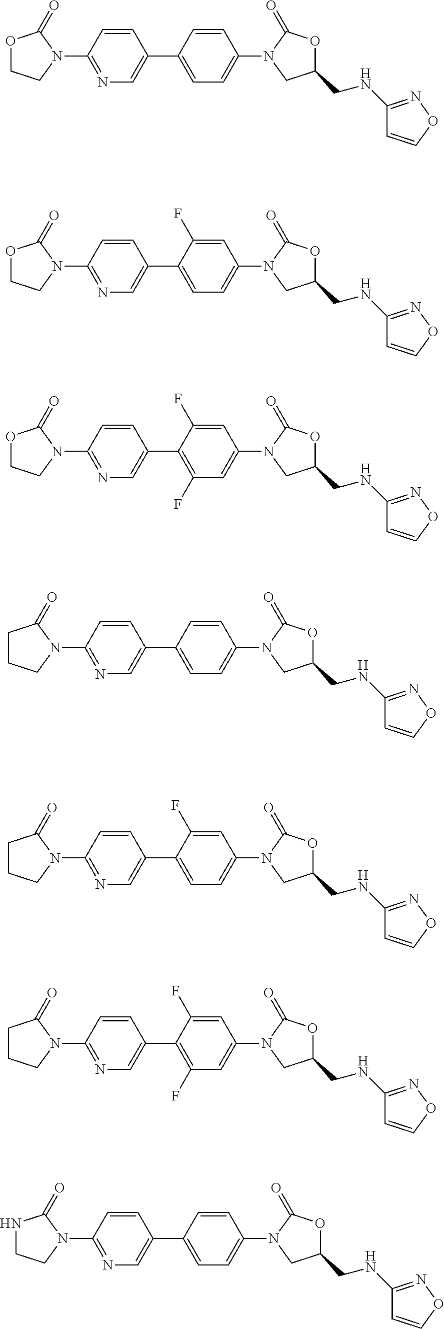

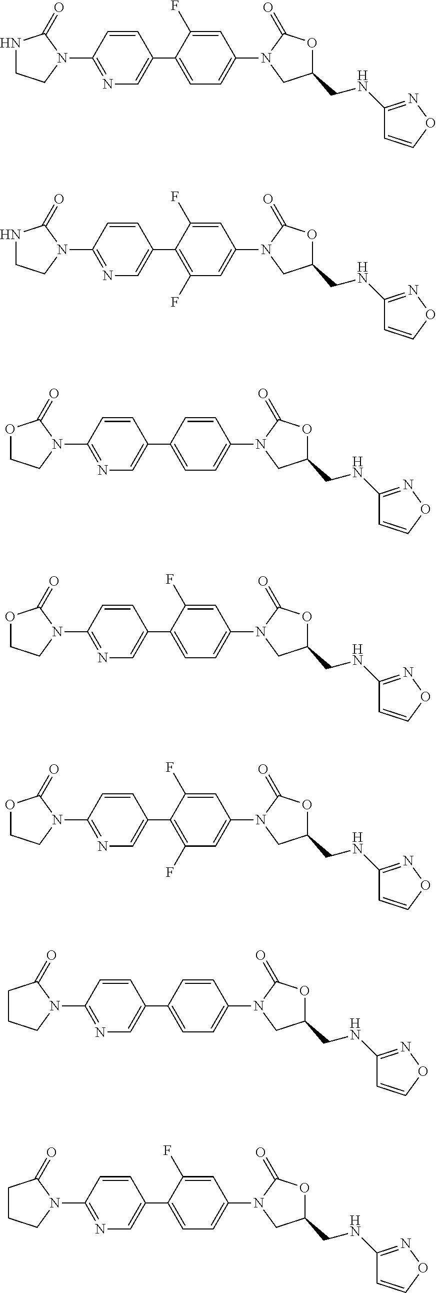

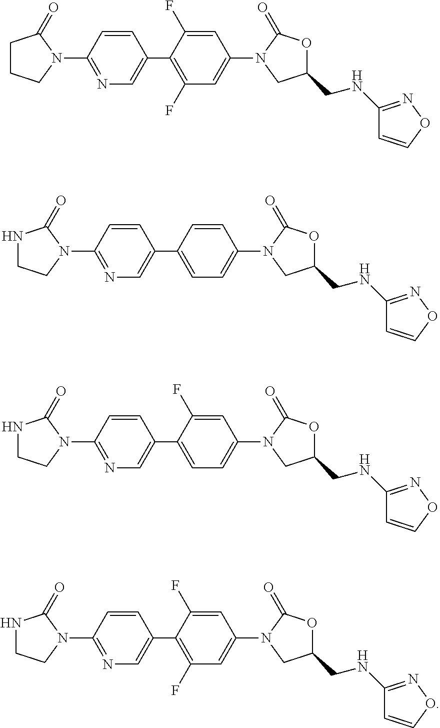

13. The compound of claim 1 selected from:

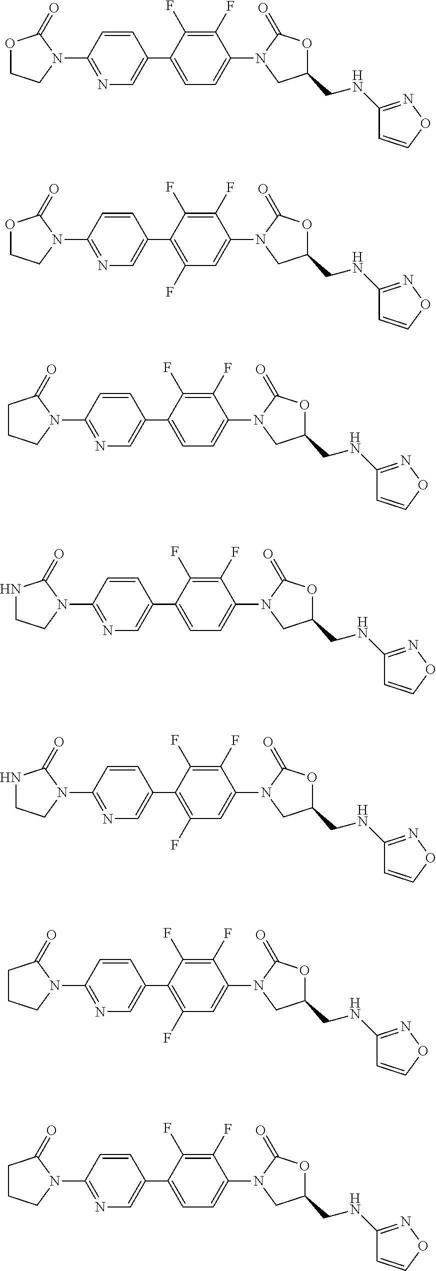

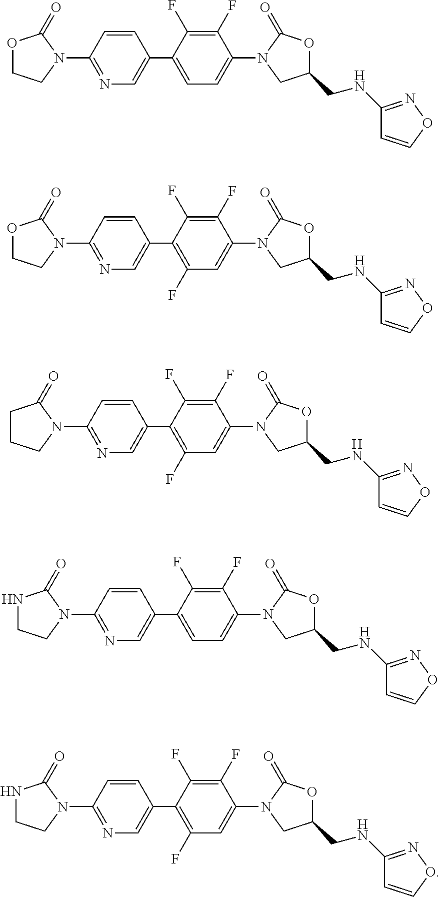

14. The compound of claim 1 selected from:

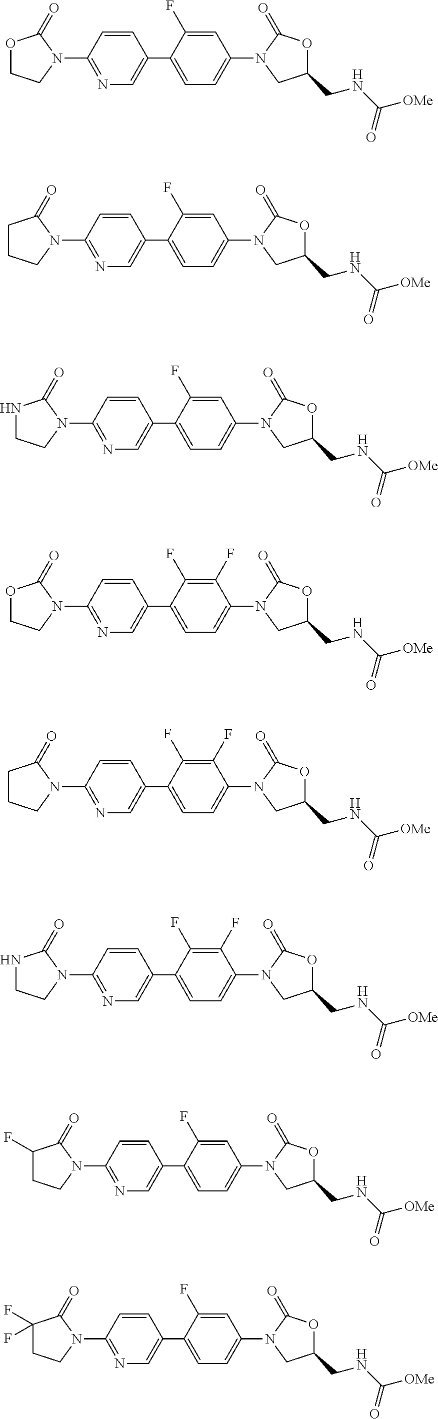

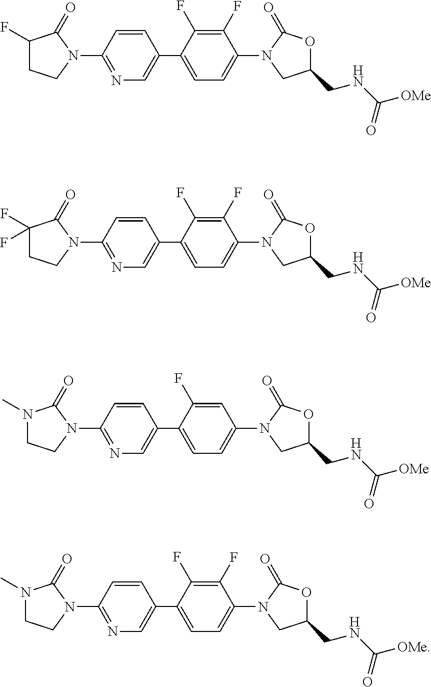

15. A method for the treatment of a microbial infection in a mammal comprising administering to the mammal a therapeutically effective amount of a compound of any of claims 1-14.

16. The method according to claim 15, wherein the compound is administered to the mammal orally, parenterally, transdermally, topically, rectally, or intranasally in a pharmaceutical composition.

17. The method according to claim 15, wherein the compound is administered once-daily in an amount of from about 1 to about 75 mg/kg of body weight/day.

18. The method according to claim 15, wherein said compound displays the minimum inhibitory concentration against linezolid-resistant Staphylococcus aureus, Enterococci faecium, or Enterococci faecalis with a value of 4 μg/mL or below.

19. The method according to claim 15, wherein said compound displays the minimum inhibitory concentration against Haemophilus influenzae with a value of 4 μg/mL or below.

20. The method according to claim 15, wherein the microbial infection is a Gram-positive microbial infection.

21. The method according to claim 15, wherein the microbial infection is a Gram-positive linezolid-resistant infection or a fastidious Gram-negative infection.

22. A pharmaceutical composition comprising a therapeutically effective amount of compound of any of claims 1-14 and a pharmaceutically acceptable carrier.
